(19) United States
(12) Patent Application Publication
(10) Pub. No.: US 2025/0051401 A1
(43) Pub. Date: Feb. 13, 2025

(54) IMPROVED PRODUCTION OF SECRETED PROTEINS IN YEAST CELLS

(71) Applicants: LESAFFRE ET COMPAGNIE, Paris (FR); EUROPEAN MOLECULAR BIOLOGY LABORATORY, Heidelberg (DE)

(72) Inventors: Thomas Desfougeres, Dissay (FR); Thierry Dulermo, Croix (FR); Georges Pignede, Marcq en Baroeul (FR); Lars Steinmetz, Heidelberg (DE); Andreas S. Johansson, Heidelberg (DE)

(21) Appl. No.: 18/717,969

(22) PCT Filed: Dec. 7, 2022
(86) PCT No.: PCT/EP2022/084829

§ 371 Date: Jun. 7, 2024

(30) Foreign Application Priority Data
Dec. 8, 2021 (EP) 21213084.3

(51) Int. Cl. C07K 14/37; C12N 1/18; C12N 9/16; C12N 9/80; C12N 15/81; C12R 1/865

(52) U.S. Cl. CPC C07K 14/37; C12N 1/185; C12N 9/16; C12N 9/80; C12N 15/81; C12N 2510/00; C12R 2001/865; C12Y 305/01098

(57) ABSTRACT

The present invention relates to a yeast cell producing at least one secreted protein of interest, wherein said cell comprises at least one additional fungal gene showing increased expression and/or overexpression, showing reduced expression and/or inactivation, wherein said gene improves the production of the at least one secreted protein of interest. The present invention further relates to respective methods for production and uses of the yeast cell.

The present invention relates to a yeast or filamentous fungal cell producing at least one secreted protein of interest, wherein said cell comprises at least one additional fungal gene showing increased expression and/or overexpression, showing reduced expression and/or inactivation, wherein said gene improves the production of the at least one secreted protein of interest. The present invention further relates to respective methods for production and uses of the yeast or filamentous fungal cells.

### BACKGROUND OF THE INVENTION

The production of recombinant enzymes is growing rapidly and is estimated to generate several tens of billions of dollars (Martinez et al., 2012). Almost 60% of the enzymes used in detergents, the food industry and biofuel alcohol are recombinant enzymes, i.e. produced by an organism other than that of origin of the protein (COWAN, 1996). The expression of enzymes in a heterologous host allows (i) the production of enzymes of interest from slow growing or even non-cultivable organisms, (ii) the much higher production of the enzyme of interest, (iii) the production of proteins from pathogenic or toxin-producing organisms, and (iv) the increase of the stability or activity of an enzyme by protein engineering (Falch, 1991; Demain and Vaishnav, 2009).

Many microorganisms, including filamentous fungi (Aspergillus sp., Trichoderma sp.), yeasts (for example Pichia pastoris, Saccharomyces cerevisiae, Yarrowia lipolytica) or bacteria (for example Escherichia coli, Bacillus sp.), are used to produce recombinant proteins (Demain and Vaishnav, 2009).

The production of recombinant proteins is dependent on the expression cassette (promoters and terminators used, signal sequence, codon bias), on the cellular machinery involved in the synthesis and degradation of proteins, intracellular trafficking and/or secretion, but also the energy level and/or redox of the cell as well as the culture conditions and the availability of nutrients (Zahrl et al., 2019).

Compared to other organisms conventionally used to produce recombinant proteins, S. cerevisiae has the advantage of rapid growth, easy manipulation both at the genetic level and at the level of production in bioreactors, and having Generally Recognized As Safe (GRAS) status. The production of a heterologous target protein in yeast host cells is further advantageous in that it allows the target proteins to be folded and secreted through the cellular secretory machinery.

Yeast is already widely used for many industrial applications (breadmaking, production of drinking alcohol and biofuels, etc. Parapouli et al., 2020) where it may be advantageous to have it produce heterologous enzymes. For example, in the field of biofuel alcohol, the commercialized yeast strains of S. cerevisiae secrete enzymatic activities allowing the degradation of industrial mashes containing starch derivatives. This allows bioethanol manufacturers to limit their intake of exogenous enzymes and reduce their production costs.

US 2011-0129872A1 relates to a method for producing a recombinant protein, comprising culturing a yeast transformed with a recombinant gene construct comprising a yeast promoter, a gene coding a signal sequence and a gene coding a target protein; and also with one or more genes coding folding accessory protein selected from the group consisting of PDI1 (protein disulfide isomerase 1), SEC23 (secretory 23), TRX2 (thioredoxin 2) AHA1 (activator of heat shock protein 90 ATPase), and SCJ1 (S. cerevisiae DnaJ), followed by culturing the transformed yeast.

US 2013-0011875 relates to a method and the production of higher titers of recombinant protein in a modified yeast host cell, for example Pichia pastoris, wherein the modified yeast cell lacks vacuolar sorting activity or has decreased vacuolar sorting activity relative to an unmodified yeast host cell of the same species.

US 2014-0335622 discloses an expression vector for secreting a protein (Z) to be recovered or a fusion protein having the protein (Z) moiety therein; a method for producing a transformant using the expression vector; the transformant; and a method for producing a protein using the transformant. It is disclosed that co-expression of a foreign secretory protein with PDI1 increases the secretory production amount.

US 2016-0186192 describes a method for producing a desired protein comprising: (a) providing a host cell comprising a first recombinant gene encoding a protein comprising the sequence of a first chaperone protein, a second recombinant gene encoding a protein comprising the sequence of a second chaperone protein and a third gene, such as a third recombinant gene, encoding a desired protein (such as a desired heterologous protein), wherein the first and second chaperones are different; and (b) culturing the host cell in a culture medium to obtain expression of the first, second and third genes.

US 2018-0022785 claims a method for producing a heterologous protein, said method comprising: culturing a Saccharomyces cerevisiae yeast host cell or a culture thereof to produce the heterologous protein, wherein said Saccharomyces cerevisiae yeast host cell comprises a modified Not4 protein, and wherein said heterologous protein is an albumin, or a variant, fragment and/or fusion thereof.

Eun Jung Thak et al. (in: Yeast synthetic biology for designed cell factories producing secretory recombinant proteins, FEMS Yeast Research, Volume 20, Issue 2, March 2020, foaa009, https://doi.org/10.1093/femsyr/foaa009) disclose that yeasts are prominent hosts for the production of recombinant proteins from industrial enzymes to therapeutic proteins. Particularly, the similarity of protein secretion pathways between these unicellular eukaryotic microorganisms and higher eukaryotic organisms has made them a preferential host to produce secretory recombinant proteins. However, there are several bottlenecks, in terms of quality and quantity, restricting their use as secretory recombinant protein production hosts. They discuss recent developments in synthetic biology approaches to constructing yeast cell factories endowed with enhanced capacities of protein folding and secretion as well as designed targeted post-translational modification process functions, and focus on the new genetic tools for optimizing secretory protein expression, such as codon-optimized synthetic genes, combinatory synthetic signal peptides and copy number-controllable integration systems, and the advanced cellular engineering strategies, including endoplasmic reticulum and protein trafficking pathway engineering, synthetic glycosylation, and cell wall engineering, for improving the quality and yield of secretory recombinant proteins.

Zihe Liu, et al. (in: Improved Production of a Heterologous Amylase in Saccharomyces cerevisiae by Inverse Metabolic Engineering, Applied and Environmental Microbiology August 2014, 80 (17) 5542-5550; DOI: 10.1128/AEM.00712-14) disclose that the increasing demand for industrial enzymes and biopharmaceutical proteins relies on robust production hosts with high protein yield and productivity. Being one of the best-studied model organisms and capable of performing posttranslational modifications, the yeast Saccharomyces cerevisiae is widely used as a cell factory for recombinant protein production. However, many recombinant proteins are produced at only 1% (or less) of the theoretical capacity due to the complexity of the secretory pathway, which has not been fully exploited. They applied the concept of inverse metabolic engineering to identify novel targets for improving protein secretion. Screening that combined UV-random mutagenesis and selection for growth on starch was performed to find mutant strains producing heterologous amylase 5-fold above the level produced by the reference strain. Genomic mutations that could be associated with higher amylase secretion were identified through whole-genome sequencing. Several single-point mutations, including an S196I point mutation in the VTA1 gene coding for a protein involved in vacuolar sorting, were evaluated by introducing these to the starting strain. By applying this modification alone, the amylase secretion could be improved by 35%. As a complement to the identification of genomic variants, transcriptome analysis was also performed in order to understand on a global level the transcriptional changes associated with the improved amylase production caused by UV mutagenesis.

Huang, M., et al. (in: Efficient protein production by yeast requires global tuning of metabolism. Nat Commun 8, 1131 (2017). https://doi.org/10.1038/s41467-017-00999-2) describe that the biotech industry relies on cell factories for production of pharmaceutical proteins, of which several are among the top-selling medicines. There is, therefore, considerable interest in improving the efficiency of protein production by cell factories. Protein secretion involves numerous intracellular processes with many underlying mechanisms still remaining unclear. They used RNA-seq to study the genome-wide transcriptional response to protein secretion in mutant yeast strains, and find that many cellular processes have to be attuned to support efficient protein secretion. In particular, altered energy metabolism resulting in reduced respiration and increased fermentation, as well as balancing of amino-acid biosynthesis and reduced thiamine biosynthesis seem to be particularly important. They confirmed their findings by inverse engineering and physiological characterization and show that by tuning metabolism cells are able to efficiently secrete recombinant proteins.

Huang M, et al. (In: Microfluidic screening and whole-genome sequencing identifies mutations associated with improved protein secretion by yeast. Proc Natl Acad Sci USA. 2015 Aug. 25; 112(34):E4689-96. doi: 10.1073/pnas.1506460112. Epub 2015 Aug. 10. PMID: 26261321; PMCID: PMC4553813) disclose that there is an increasing demand for biotech-based production of recombinant proteins for use as pharmaceuticals in the food and feed industry and in industrial applications, that the yeast Saccharomyces cerevisiae is among preferred cell factories for recombinant protein production, and there is increasing interest in improving its protein secretion capacity. Due to the complexity of the secretory machinery in eukaryotic cells, it is said to be difficult to apply rational engineering for construction of improved strains. They used high-throughput microfluidics for the screening of yeast libraries, generated by UV mutagenesis. Several screening and sorting rounds resulted in the selection of eight yeast clones with significantly improved secretion of recombinant α-amylase. Efficient secretion was genetically stable in the selected clones. They performed whole-genome sequencing of the eight clones and identified 330 mutations in total. Gene ontology analysis of mutated genes revealed many biological processes, including some that had not been identified before in the context of protein secretion. Mutated genes identified are disclosed to be potentially used for reverse metabolic engineering, with the objective to construct efficient cell factories for protein secretion. The combined use of microfluidics screening and whole-genome sequencing to map the mutations associated with the improved phenotype can easily be adapted for other products and cell types to identify novel engineering targets, and this approach could broadly facilitate design of novel cell factories.

Bao et al. (in: Moderate Expression of SEC16 Increases Protein Secretion by Saccharomyces cerevisiae, APPLIED AND ENVIRONMENTAL MICROBIOLOGY, vol. 83, no. 14, 15 Jul. 2017) discloses that a moderate overexpression of the gene SEC16 increases protein secretion by S. cerevisiae. SEC16 is involved in protein translocation from the endoplasmic reticulum to the Golgi apparatus. The data also show that a high-level expression of SEC76 could be harmful for the cell due to higher accumulation of reactive oxygen species (ROS) and thus for recombinant protein production. Qi et al (in: Different Routes of Protein Folding Contribute to Improved Protein Production in Saccharomyces cerevisiae, mBio, 10 Nov. 2020 (2020-11-10), xP055932697, Retrieved from the Internet: URL:https://doi.org/10.1128/mBio 0.02743-20) discloses that overexpression of Cwh41p improves protein production as seen by an increased α-amylase productivity.

WO2019027364 discloses recombinant S. cerevisiae allowing increased production of secreted proteins. It is suggested to overexpress PDI1 and Sec3, and/or downregulate the expression of YPS7, and VSP27.

WO200607511 discloses the use of chaperones to improve the production of a desired protein (secreted). One chaperone used is CCT3. JP2009240185 discloses the promotion of protein production by disrupting for example the VHS2 gene or the VSP27. WO094/08024 discloses recombinant yeast and filamentous fungi transformed with SSO genes, showing increased capacity to produce secreted foreign or endogenous proteins.

Finally, Huang M, et al. (in: Engineering the protein secretory pathway of Saccharomyces cerevisiae enables improved protein production. Proc Natl Acad Sci USA. 2018 Nov. 20; 115(47):E11025-E11032. doi: 10.1073/pnas.1809921115. Epub 2018 Nov. 5. PMID: 30397111; PMCID: PMC6255153) describe that baker's yeast Saccharomyces cerevisiae is one of the most important and widely used cell factories for recombinant protein production. Many strategies have been applied to engineer this yeast for improving its protein production capacity, but productivity is still relatively low, and with increasing market demand, it is important to identify new gene targets, especially targets that have synergistic effects with previously identified targets. Despite improved protein production, previous studies rarely focused on processes associated with intracellular protein retention. They identified genetic modifications involved in the secretory and trafficking pathways, the histone deacetylase complex, and carbohydrate metabolic processes as targets for improving protein secretion in yeast. Especially modifications of endosome-to-Golgi trafficking was found to effectively reduce protein retention besides increasing protein secretion. Through combinatorial genetic manipulations of several of the newly identified gene targets, they enhanced the protein production capacity of yeast by more than fivefold, and the best engineered strains could produce 2.5 g/L of a fungal α-amylase with less than 10% of the recombinant protein retained within the cells, using fed-batch cultivation.

Cryptic unstable transcripts (CUTs) are a subset of non-coding RNAs (ncRNAs) that are produced from intergenic and intragenic regions. Additionally, stable uncharacterized transcripts, or SUTs, have also been detected in cells and bear many similarities to CUTs but are not degraded through the same pathways.

Genetic engineering strategies to overcome bottlenecks in the yeast protein secretion pathway have to consider that protein secretion in yeast involves multiple complex steps, such as protein translocation, folding, post-translational modification and vesicle trafficking between several membrane organelles and plasma membranes. The secretion of proteins synthesized inside cells can be hampered by low secretion efficiency, abnormal post-translational modifications, retention within the secretion pathway or the cell wall space as a cell-associated form. The development of engineering strategies targeted to each step of the secretion pathway in a modular fashion is required in order to design cell factories producing secretory recombinant proteins. Today, despite its obvious qualities, S. cerevisiae remains relatively limited in its ability to secrete proteins compared to organisms such as filamentous fungi or P. pastoris (Demain and Vaishnav, 2009). It is therefore an object of the present invention to provide new factors to improve recombinant protein production and secretion in yeast. Other objects and advantages will become apparent to the person of skill when studying the present description of the present invention.

In a first aspect of the present invention, the above object is solved in accordance with the claims, preferably by providing a cell of Saccharomyces cerevisiae, producing at least one secreted protein of interest, wherein said cell comprises at least one fungal gene selected from the group consisting of ENO2, NMA2, PRY2, SUT074, TFG2, AVT2, TRM10, BNA7, and TOM22, wherein said at least one fungal gene shows increased expression and/or overexpression, and/or wherein said cell comprises at least one fungal gene selected from the group consisting of TLG2, MNT2, TPO2, ATG33, THR4, INP51, CUT901, YDR262W, MRP10, NDC1, and CMC1, wherein said at least one fungal gene shows reduced expression and/or inactivation, and optionally further comprising the fungal gene HDA2 and/or PDI1, showing an increased expression and/or overexpression.

Preferred is the yeast cell according to the present invention, wherein said cell comprises at least one fungal gene selected from the groups consisting of ENO2, NMA2, PRY2, SUT074, and TFG2, or AVT2, TRM10, PRY2, SUT074, BNA7, and TOM22, wherein said at least one fungal gene shows increased expression and/or overexpression, and/or wherein said cell comprises at least one fungal gene selected from the groups consisting of TLG2, CUT901, ATG33, THR4, YDR262W, and CMC1, or MRP10, TLG2, CUT901, ATG33, THR4, YDR262W, CMC1, MNT2, TPO2, and NDC1, preferably MNT2 and TPO2, wherein said at least one fungal gene shows reduced expression and/or inactivation, and optionally further comprising the fungal genes HDA2 and/or PDI1, showing an increased expression and/or overexpression, and/or INP51 showing an reduced expression and/or inactivation.

The above object is further solved according to the present invention by providing a yeast or filamentous fungal cell producing at least one secreted protein of interest, wherein said cell comprises at least one fungal gene selected from the group consisting of MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL11, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, wherein said at least one fungal gene shows increased expression and/or overexpression, and/or wherein said cell comprises at least one fungal gene selected from the group consisting of TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, CUT729, INP51, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, wherein said at least one fungal gene shows reduced expression and/or inactivation.

Preferably, said cell comprises at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MAL11, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

More preferred is the yeast or filamentous fungal cell according to the present invention, wherein said genes or SUTs or CUTs are furthermore selected from the group of genes or SUTs or CUTs having a value of log FC/FDR log FC/FDR of more than 40, preferably of more than 200, more preferred of more than 300, and most preferred of more than 500, based on the values as determined herein.

More preferred is the yeast or filamentous fungal cell according to the present invention, further comprising a fungal gene selected from the group consisting of THR4, MRP10, RIP1, YLR342W-A, ATG33, and YOR238W, either showing an increased expression and/or overexpression or reduced expression and/or inactivation, depending on the experimental conditions, such as, without wanting to be bound by theory, for example, the impact of CRISPRa and CRISPRi on gene expression due to the position of the gRNA in the promoting region.

Even more preferred is the yeast or filamentous fungal cell according to the present invention, further comprising the fungal gene HDA2 and/or PDI1, showing an increased expression and/or overexpression.

Advantageously, the yeast or filamentous fungal cell according to the present invention produces the at least one secreted protein to about 20% or more about, or about 30% or more, or about 40% or more, preferably about 50% or more, more preferably to about 75% or more, when compared to a control yeast or filamentous fungal cell.

In a second aspect of the present invention, the above object is solved by a method for producing a secreted protein in a yeast or filamentous fungal cell, comprising the steps of i) providing a yeast or filamentous fungal cell producing at least one secreted protein of interest according to the present invention, ii) culturing said yeast or filamentous fungal cell in suitable culture medium, and iii) isolating said secreted protein from aid culture medium. Preferred is the method according to the present invention, further comprising suitably inducing the increased expression and/or overexpression or reduced expression and/or inactivation of the at least one fungal gene.

Further preferred is the method according to the present invention, wherein about 30% or more, or about 40% or more, preferably about 50% or more, more preferably to about 75% or more of said at least one secreted protein is produced, when compared to the production of a control yeast or filamentous fungal cell.

In a third aspect of the present invention, the above object is solved by a method for producing a yeast or filamentous fungal cell producing at least one secreted protein of interest according to the present invention, comprising introducing into said cell producing at least one secreted protein of interest at least one fungal gene selected from the group consisting of MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL11, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, wherein said at least one fungal gene shows increased expression and/or overexpression, and/or wherein said cell comprises at least one fungal gene selected from the group consisting of TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, INP51, CUT729, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, wherein said at least one fungal gene shows reduced expression and/or inactivation.

Preferred is a method of the present invention for producing a yeast or filamentous fungal cell producing at least one secreted protein of interest according to the present invention, comprising introducing into said cell producing at least one secreted protein of interest at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MAL11, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

Furthermore, the method according to the invention may include further introducing into said cell a fungal gene selected from the group consisting of THR4, MRP10, RIP1, YLR342W-A, ATG33, and YOR238W, either showing an increased expression and/or overexpression or reduced expression and/or inactivation, depending on the experimental conditions. Furthermore, the method may include further introducing into said cell the fungal gene HDA2 and/or PDI1, showing an increased expression and/or overexpression.

In a fourth aspect of the present invention, the above object is solved by the use of a yeast or filamentous fungal cell according to the present invention for producing at least one secreted protein of interest.

As mentioned above, the analysis of UV S. cerevisiae mutants expressing an α-amylase has revealed improved strains for secretion (Huang et al., 2015; Huang et al., 2018). Coupling microfluidics with a phenotypic screening using a starch complexed with BODIPY (which becomes fluorescent when it is released), the authors had selected the mutants secreting the most enzyme into the extracellular medium. The sequencing of eight hypersecretory clones (×1.5 to ×6) revealed 330 mutations potentially involved in improving α-amylase production and secretion (Huang et al., 2015). A more in-depth analysis led to the identification of—amongst others as disclosed herein—a role of the known PDI1 gene in the production and secretion of α-amylase in S. cerevisiae.

The purpose of the present invention was to discover new factors and genes involved in protein secretion in order to improve protein production and secretion, as exemplified in the industrial Ethanol Red® strain of S. cerevisiae.

As mentioned above, in the first aspect of the present invention, a yeast or filamentous fungal cell is provided that produces at least one secreted protein of interest. In addition, the cell comprises at least one fungal gene selected from the group consisting of MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL11, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, wherein these at least one fungal gene shows increased expression and/or overexpression.

In the context of the present invention, the terms “increased expression” or “overexpression” indicate that the amount of protein as produced by the cell is higher when compared to the expression in a control cell showing normal, unaltered or baseline expression. The change in expression can be achieved in any suitable way, and examples include mutated promotors, cloning of the gene under the control of a heterologous “strong” promotor, either inducible or constitutive, codon optimization, and mutations that stabilize the structure of the protein, and the like. In the context of the present invention, a preferred example of how to detect “increased expression” or “overexpression” is a change in log FC (log fold change, see the tables below), more preferably a statistically relevant change (FDR) in the log FC. Examples are a value of log FC/FDR of more than 40, preferably of more than 200, more preferred of more than 300, and most preferred of more than 500, based on the values as determined herein.

Alternatively or in addition, the cell comprises at least one fungal gene selected from the group consisting of TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, INP51, CUT729, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, wherein said at least one fungal gene shows reduced expression and/or inactivation, wherein said at least one fungal gene shows reduced expression and/or inactivation. In the context of the present invention, the terms “reduced expression” or “inactivation” indicate that the amount of protein as produced by the cell is lower when compared to the expression in a control cell showing normal, unaltered or baseline expression. The change in expression can be achieved in any suitable way, and examples include mutated promotors, cloning of the gene under the control of a heterologous “weak” promotor, either inducible or constitutive, codon changes, and mutations that de-stabilize the structure of the protein, and the like.

Systematic studies of the effects on protein secretion from gene perturbations are challenging, primarily due to the size of the readout, yeast encodes around 6300 genes, in addition to other genetic elements, including long non-coding RNAs, such as cryptic untranslated transcripts (CUTs) and stable uncharacterized transcripts (SUTs) that are not transcribed into proteins, but instead affect and modulate gene expression in the nucleus or the cytosol.

Preferably, said yeast or filamentous fungal cell as provided comprises at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MAL11, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

More preferred is the yeast or filamentous fungal cell according to the present invention, wherein said genes or SUTs or CUTs are furthermore selected from the group of genes or SUTs or CUTs having a value of log FC/FDR of more than 40, preferably of more than 200, more preferred of more than 300, and most preferred of more than 500, based on the values as determined herein.

More preferred is the yeast or filamentous fungal cell according to the present invention, further comprising a fungal gene selected from the group consisting of THR4, MRP10, RIP1, YLR342W-A, ATG33, and YOR238W, either showing an increased expression and/or overexpression or reduced expression and/or inactivation, depending on the experimental conditions.

Even more preferred is the yeast or filamentous fungal cell according to the present invention, further comprising the fungal gene HDA2 and/or PDI1, showing an increased expression and/or overexpression.

It is expected that a combination of genes as mentioned herein can lead to an even further increased production of the protein of interest, even having synergistic effects. Examples for these combinations are all of TLG2, YDR262W, and TRM10, optionally further comprising HDA2 and/or PDI1. Other examples are ATG33 and MRP10, NDC1 and TRM10, or PRY2, and TOM22, again each pair optionally further comprising HDA2 and/or PDI1.

Most preferred are either AVT2, PRY2, SUT074, BNA7, TOM22 or TRM10. The overexpression of AVT2, TRM10, PRY2, SUT074, BNA7, or TOM22, and the inactivation of INP51 is further preferred. Further examples are TLG2, CUT901, ATG33, THR4, YDR262W, and CMC1, optionally further comprising HDA2 and/or PDI1. Also preferred is ENO2, NMA2, PRY2, SUT074, and TFG2 (increased expression and/or overexpression), MNT2, and TPO2 (reduced expression and/or inactivation), optionally further comprising HDA2 and/or PDI1.

The fungal gene(s) and/or SUTs or CUTs as used are preferably derived from S. cerevisiae, or a related yeast. The fungal gene(s) and/or SUTs or CUTs and their reference numbers are according to the Saccharomyces Genome Database (SGD) (https://www.yeastgenome.org/), as of Nov. 15, 2021. Related genes that may be used as well encode for proteins sharing the same biological effect (increased secretion) in the yeast or filamentous fungal cell with the genes as above, and/or have an amino acid identity of about 80% or more, preferably about 90% or more, more preferably about 95% or more with the polypeptide sequence as encoded by a genes as above.

Advantageously, preferably the yeast or filamentous fungal cell according to the present invention produces the at least one secreted protein to about 30% or more or 40% or more, preferably about 50% or more, more preferably to about 75% or more, when compared to a control yeast or filamentous fungal cell, preferably one that does not contain a gene as mentioned above leading to increased secretion of the protein of interest.

As the protein of interest, any protein can be chosen that can be suitably produced by the yeast or filamentous fungal cell according to the present invention, e.g. expressed, folded, glycosylated and/or secreted. The gene of the protein of interest can be codon optimized, and preferably show an increased expression and/or overexpression, as explained above for the fungal gene according to the present invention. Examples of preferred proteins of interest are human serum albumin (HSA), amylase, human insulin, and components of hepatitis vaccines, human papillomavirus (HPV) vaccines, interferon(s), or epidermal growth factor (hEGF), and proteins used in food production, such as cellulase, glucoamylase, xylanase, and the like.

In order to identify new genes involved in the production and secretion of recombinant and heterologous proteins in yeast or filamentous fungal cells, such as S. cerevisiae, the inventors have developed CRISPRi and CRISPRa libraries allowing the overexpression or the repression of all genes as well as previously identified Stable Unannotated Transcripts (SUT's) and (Cryptic Unstable Transcripts CUT's) of this yeast (see Xu, Z. et al. Bidirectional promoters generate pervasive transcription in yeast. Nature 457, 1033-1037 (2009)). These libraries utilize an inactivated Cas9 (dCas9) able to bind DNA at the CRISPR site but unable to cleave the DNA molecule, fused to a transcriptional activation (CRISPRa) (e.g. the VP64-p65-Rta (VPR) tripartite activation domain described in Chavez, A. et al. Highly efficient Cas9-mediated transcriptional programming. Nat Methods 12, 326-328 (2015)) or repression domain (CRISPRi) (Dominguez et al., 2015).

The industrial Ethanol Red® (ER) yeast strain overexpressing an α-amylase (Amy6 from A. niger) was used as a model for the present invention (Lesaffre, Marcq-en-Baroeul, France). A 40,890 gRNA library targeting the promoters of 7,247 yeast genes, SUT's and CUT's at an average of 5.8 positions per gene, SUT or CUT was developed and cloned into replicative vectors allowing their expression as well as the expression of dCas9-VP64-p65-Rta (CRISPRa) or dCas9-Mxi1 (CRISPRi). The ER+α-amylase strain was then transformed using the CRISPRa and CRISPRi libraries, and the cell population as obtained was screened by microfluidics on the basis of its capacity to degrade a starch substrate labelled with BODIPY FL dye which fluoresces in green when the starch is degraded by α-amylase (e.g. EnzChek® Ultra Amylase Assay Kit: https://www.thermofisher.com/order/catalog/product/E33651 #/E33651).

Clones presenting high fluorescence were sorted, and gRNA regions from replicative vectors were analyzed by Illumina sequencing. Data analysis revealed that 320 activated or repressed genes favor α-amylase secretion. These genes were manually selected further, and the genes MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL1I, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, were overexpressed using common techniques (integration of overexpression cassette into the genome and/or overexpression through a replicative plasmid), and genes TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, INP51, CUT729, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, were inactivated by gene deletion. Then, α-amylase activity was evaluated in the respective strains. The overexpression of BNA7, SUT074, TOM22, TLG2, YDR262W, ALP1, ENO2, NMA2, PRY2, and INP51 were identified as preferred for the exemplary α-amylase secretion in the Ethanol Red® strain.

In the context of the present invention, any suitable cell of a yeast or filamentous fungus can be used for the production of the protein of interest according to the present invention. Preferably, said yeast or filamentous fungal cell is selected from the group consisting of Aspergillus spp., Trichoderma spp., Saccharomyces cerevisiae, Schizosaccharomyces pombe, Kluyveromyces ssp., Pichia spp., Hansenula polymorpha, Fusarium spp., Neurospora spp., and Penicillium spp., preferably Saccharomyces cerevisiae.

In the yeast or filamentous fungal cell according to the present invention, the at least one fungal gene showing increased expression and/or overexpression and/or showing reduced expression and/or inactivation is a native gene and/or is a recombinant gene, i.e. a modified gene of the yeast or filamentous fungal cell itself, or at least one gene that is recombinantly introduced and may be a heterologous gene, i.e. coming from a different strain or fungal species. Preferably, the recombinant gene is integrated into the genome as an expression cassette. Respective expression cassettes for fungal expression are known, and basically consist of a promoter, the fungal gene, and a terminator. Alternatively or in additionally, the gene can be extrachromosomally expressed, preferably using a replicative expression vector, such as a shuttle vector. Promoters used in yeast and fungal expression systems are usually either inducible or constitutive.

The folding and glycosylation of the secretory proteins in the endoplasmatic reticulum (ENDR) is assisted by numerous ENDR-resident proteins. The chaperones like Bip (GRP78), GRP94 or yeast Lhslp help the secretory protein to fold by binding to exposed hydrophobic regions in the unfolded states and preventing unfavourable interactions (Blond-Elguindi et al., 1993, Cell 75:717-728). The chaperones are also important for the translocation of the proteins through the ENDR membrane. The proteins like protein disulphide isomerase and its homologs and prolyl-peptidyl cis-trans isomerase assist in formation of disulphide bridges and formation of the right conformation of the peptide chain adjacent to proline residues, respectively. A machinery including many protein components also resides in the ENDR for the addition of the N-linked core glycans to the secretory protein and for the initial trimming steps of the glycans.

Preferred is therefore the yeast or filamentous fungal cell according to the present invention, wherein the cell furthermore comprises at least one additional recombinant secretion promoting gene, for example a fungal gene for a chaperone, for a foldase and/or for a glycosylation-promoting protein. Like the other genes as disclosed herein, these proteins may be controllably expressed, inducible, constitutive, and even overexpressed.

Therefore, preferred is the yeast or filamentous fungal cell according to the present invention, wherein the increased expression and/or overexpression or reduced expression and/or inactivation of the at least one fungal gene or the at least one additional recombinant secretion promoting gene is constitutive or inducible.

Another important aspect of the present invention relates to a method for producing a secreted protein in a yeast or filamentous fungal cell, comprising the steps of i) providing a yeast or filamentous fungal cell producing at least one secreted protein of interest according to the present invention as above, ii) suitably culturing said yeast or filamentous fungal cell in suitable culture medium, and iii) isolating said secreted protein from said culture medium. Methods for isolating proteins from cultures are known by the person of skill.

Culturing methods for producing proteins in yeast or filamentous fungal cells are known by the person of skill, and can be readily adjusted to the present invention. Culturing can be continuous or in batches or fed-batches. Preferred is the method according to the present invention, further comprising suitably inducing the increased expression and/or overexpression or reduced expression and/or inactivation of the at least one fungal gene. Induction can be achieved based on the promotor(s) as used, e.g. by adding inducers, or switching conditions, e.g. temperature.

There are many examples of engineering of S. cerevisiae for improved protein production, including optimizing of fermentation process, selecting the expression vectors systems, choosing the signal sequence for extracellular targeting and engineering host strains for better folding and post-translational modification (Tohda H., Kumagai H., Takegawa, K, (2010) Engineering of protein secretion in yeast: strategies and impact on protein production. Appl Microbiol Biotechnol 86: 403-417).

Preferred is the method according to the present invention, wherein about 30% or more or 40% or more, preferably about 50% or more, more preferably to about 75% or more of said at least one secreted protein is produced, when compared to the production of a control yeast or filamentous fungal cell, preferably one that does not contain a gene as mentioned above leading to increased secretion of the protein of interest

Another important aspect of the present invention relates to a method for producing a yeast or filamentous fungal cell producing at least one secreted protein of interest, comprising introducing into said cell producing at least one secreted protein of interest at least one fungal gene selected from the group consisting of MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL11, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, wherein said at least one fungal gene shows increased expression and/or overexpression, and/or wherein said cell comprises at least one fungal gene selected from the group consisting of TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, INP51, CUT729, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, wherein said at least one fungal gene shows reduced expression and/or inactivation. Preferably, said at least one fungal gene is integrated into the genome as an expression cassette and/or extrachromosomally expressed, preferably using a replicative expression vector.

Preferred is a method of the present invention for producing a yeast or filamentous fungal cell producing at least one secreted protein of interest according to the present invention, comprising introducing into said cell producing at least one secreted protein of interest at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MAL11, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

Furthermore, the method according to the invention may include further introducing into said cell a fungal gene selected from the group consisting of THR4, MRP10, RIP1, YLR342W-A, ATG33, and YOR238W, either showing an increased expression and/or overexpression or reduced expression and/or inactivation, depending on the experimental conditions.

In a preferred embodiment according to the method according to the present invention, said method further comprises introducing into said cell the fungal gene HDA2 and/or PDI1, showing an increased expression and/or overexpression. Preferably, said at least one fungal gene is also integrated into the genome as an expression cassette and/or extrachromosomally expressed, preferably using a replicative expression vector

Finally, another important aspect of the present invention relates to the use of a yeast or filamentous fungal cell according to the present invention for producing at least one secreted protein of interest, preferably using a method according to the present invention.

In the context of the present invention, the inventors deploy genome-wide CRISPRi (repression, Smith, J. D. et al. Quantitative CRISPR interference screens in yeast identify chemical-genetic interactions and new rules for guide RNA design. Genome Biol 17, 45 (2016)) and CRISPRa (activation, Chavez, A. et al. Highly efficient Cas9-mediated transcriptional programming. Nat Methods 12, 326-328 (2015)) libraries to systematically probe the effects from perturbations of gene expression on the protein secretion machinery; by targeting the transcription of all identified genes, SUT's and CUTs in S. cerevisiae on a per gene basis. The application of CRISPR/Cas9 in combination with high throughput screening and next-generation sequencing (NGS) allowed the inventors to maintain a genome-wide scope with single gene precision. This is, to the inventor's knowledge, the first systematic attempt at interrogating the effects from gene activation and repression on the protein secretion machinery across all genes in yeast.

In summary, the present invention provides the following items.

Item 1. A yeast or filamentous fungal cell producing at least one secreted protein of interest, wherein said cell comprises at least one fungal gene selected from the group consisting of MIC19, TOM22, NKP1, DML1, CUT859, GAL80, APM3, COQ10, BLM10, MDH1, VHS2, ASA1, TRP4, YPS7, CUT824, YOR318C, PRM7, ERV46, FIT2, GPM3, CUT892, SRN2, SUT643, CUT461, THR4, GMH1, SOL1, NAB6, YPR148C, ALP1, CUT097, ATG33, YOR316C-A, SOG2, MCM6, SUT230, SUT419, TIF11, TAF5, PHO91, AIM32, ENO2, UBA2, PUS5, ERG1, SUT311, KSS1, MRP10, CUT598, CUT188, YOR238W, EMW1, BNA7, SNR63, CCT3, PRY2, MAL11, KRS1 RAIl, SUT784, YPR148C, YEL1, CUT832, NMA2, VPS27, SUT428, PEX29, YLR446W, WBP1, AVT2, CUT854, TRM10, SLX9, YPL077C, PET122, TFG2, PUN1, CUT152, AIR2, CUT571, RPS26B, RRT6, RPC19, URA3, YGR045C, SMC3, PNG1, THI6, MEU1, CUT239, NSE4, SUT074, AAH1, RMD5, CUT607, ACS1, MNN1, ARH1, YHR140W, CET1, RRB1, YLR342W-A, RPS22B, CHS5, YIL165C, SUT093, LPX1, NCA3, EFG1, NBP35, CUT765, MSL1, SCD6, ATG42, CHS6, COQ2, RPO31, MKK1, HED1, PBP2, BET5, CUT678, YGR021W, SUT474, YGL159W, IRC21, VHR1, SPP1, PRP43, ZRT1, YLR041W, SUT711, COX18, CBP6, SUT575, CLG1, CUT213, QCR10, SNR3, MSS2, CUT505, YOS1, SUT073, UTP21, ACA1, CUT632, RIP1, HUL5, CUT727, RPL35B, CUT184, CUT420, YFLO41W-A, SUT460, ATG10, MFA1, UGX2, TRK2, CUT704, SUT083, TRE1, RVS161, LEAl, EBP2, THI80, CTI6, CUT322, XPT1, MRPL35, YPL025C, SUT737, PGA2, ULP2, MRX16, EST1, NUP100, IES3, ATG39, YMR084W, SUT428, YPL119C-A, MIN8, CUT490, SUT287, KEL3, SUT678, SEC3, SOL4, SIS2, CUT915, RRP3, ESA1, PCL8, TRX3, YKL115C, EMP65, ZDS1, CUT167, SOD1, UBR2, LSP1, SNR81, RGD3, YTP1, SMY2, CUT449, FIN1, YKL106C-A, YAR019W-A, CCH1, AYR1, SUT573, VNX1, FOL3, SUT511, GIS4, CUT743, RPL24A, HMT1, SUT333, SPP2, SUT128, SMC6, PHR1, RPS15, CUT642, GYP7, tK(CUU)K, CUT896, SLM5, CUT586, CUT158, CUT276, CUT480, SUT751, SUT251, CUT643, and RRP12, wherein said at least one fungal gene shows increased expression and/or overexpression, and/or wherein said cell comprises at least one fungal gene selected from the group consisting of TLG2, CUT901, ATG33, THR4, YDR262W, CMC1, MRP17, YPT52, CUT312, MRPS5, RDR1, DAL7, RPL20A, YBR137W, RPL36B, YEL008C-A, RAX1, INP51, CUT729, UBP8, CUT258, YLR342W-A, SUT568, PEX7, MSD1, CUT136, TIM10, CUT361, snR51, TAL1, RIP1, MRP10, SUT078, MRP51, GLO3, EHD3, HER1, NMA111, PBP4, MFB1, IKI3, NDL1, SUT433, YOR238W, SUT750, QDR2, RDI1, SUT014, CUT437, MSC6, SUT497, YCR051W, MRPL33, RPL14A, TRM7, RNH202, RTC5, SUT027, CDC5, SUT729, YOR131C, CUT665, GLG2, SUT268, SUT705, MED4, RCR2, EFB1, RXT2, KGD1, TUP1, RNH203, YDR338C, SED1, CUT522, HIS2, SUT145, MET17, APC4, NKP2, MKK2, NDC1, PET100, NIP7, VHT1, SUT685, BNI5, SNA3, EGH1, MRP4, POB3, PIB2, SUT317, YKL024C, YGL116W, YLR118C, YFR031C-A, YGL190C, YDL108W, YMR128W, YBR253W, YJR113C, YILO31W, YGR109C, YBR282W, YMR125W, YMR236W, YDR411C, YML029W, YDL033C, YPL050C, YHR171W, YDR352W, and NTO1, wherein said at least one fungal gene shows reduced expression and/or inactivation.

Item 2. The yeast or filamentous fungal cell according to Item 1, wherein said cell comprises at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MALI1, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

Item 3. The yeast or filamentous fungal cell according to Item 1 or 2, wherein said genes or SUTs or CUTs are furthermore selected from the group of genes or SUTs or CUTs having a value of log FC/FDR of more than 40, preferably of more than 200, more preferred of more than 300, and most preferred of more than 500, based on the values as determined herein.

Item 4. The yeast or filamentous fungal cell according to any one of Items 1 to 3, further comprising a fungal gene selected from the group consisting of THR4, MRP10, RIP1, YLR342W-A, ATG33, and YOR238W, either showing an increased expression and/or overexpression or reduced expression and/or inactivation, depending on the experimental conditions.

Item 5. The yeast or filamentous fungal cell according to any one of Items 1 to 4, further comprising the fungal gene HDA2 and/or PDI1, showing an increased expression and/or overexpression.

Item 6. The yeast or filamentous fungal cell according to any one of Items 1 to 5, wherein said yeast or filamentous fungal cell is selected from the group consisting of Aspergillus spp., Trichoderma spp., Saccharomyces cerevisiae, Schizosaccharomyces pombe, Kluyveromyces ssp., Pichia spp., Hansenula polymorpha, Fusarium spp., Neurospora spp., and Penicillium spp., preferably Saccharomyces cerevisiae.

Item 7. The yeast or filamentous fungal cell according to any one of Items 1 to 6, wherein said at least one secreted protein of interest also shows an increased expression and/or overexpression.

Item 8. The yeast or filamentous fungal cell according to any one of Items 1 to 7, wherein said at least one fungal gene showing increased expression and/or overexpression and/or showing reduced expression and/or inactivation is a native gene and/or is a recombinant gene, wherein preferably said recombinant gene is integrated into the genome as an expression cassette and/or extrachromosomally expressed, preferably using a replicative expression vector.

Item 9. The yeast or filamentous fungal cell according to any one of Items 1 to 8, wherein the cell furthermore comprises at least one additional recombinant secretion promoting gene, for example a gene for a chaperone, for a foldase and/or for a glycosylation-promoting protein.

Item 10. The yeast or filamentous fungal cell according to any one of Items 1 to 9, wherein the increased expression and/or overexpression or reduced expression and/or inactivation of the at least one fungal gene or the at least one additional recombinant secretion promoting gene is constitutive or inducible.

Item 11. The yeast or filamentous fungal cell according to any one of Items 1 to 10, wherein the cell produces the at least one secreted protein to about 30% or more, or to about 40% or more, preferably about 50% or more, more preferably to about 75% or more, when compared to a control yeast or filamentous fungal cell.

Item 12. A method for producing a secreted protein in a yeast or filamentous fungal cell, comprising the steps of i) providing a yeast or filamentous fungal cell producing at least one secreted protein of interest according to any one of Items 1 to 11, ii) culturing said yeast or filamentous fungal cell in suitable culture medium, and iii) isolating said secreted protein from said culture medium.

Item 13. The method according to Item 12, further comprising suitably inducing the increased expression and/or overexpression or reduced expression and/or inactivation of the at least one fungal gene.

Item 14. The method according to Item 11 or 12, wherein about 30% or more, or about 40% or more, preferably about 50% or more, more preferably to about 75% or more of said at least one secreted protein is produced, when compared to the production of a control yeast or filamentous fungal cell.

Item 15. A method for producing a yeast or filamentous fungal cell producing at least one secreted protein of interest, comprising introducing into said cell producing at least one secreted protein of interest at least one fungal gene selected from the group consisting of MIC19, TOM22, NKP1, DML1, CUT859, GAL80, APM3, COQ10, BLM10, MDH1, EMW1, BNA7, SNR63, CCT3, PRY2, MAL1i, KRS1, RAIl, SUT784, YPR148C, YEL1, CUT832, NMA2, VPS27, SUT428, PEX29, YLR446W, and WBP1, preferably ENO2, NMA2, PRY2, SUT074, and TFG2, wherein said at least one fungal gene shows increased expression and/or overexpression, and/or wherein said cell comprises at least one fungal gene selected from the group consisting of TLG2, CUT901, ATG33, THR4, NDC1, PET100, NIP7, VHT1, and SUT685, preferably MNT2, and TPO2, wherein said at least one fungal gene shows reduced expression and/or inactivation.

Item 16. The method according to Item 15, further introducing into said cell a fungal gene selected from the group consisting of THR4, MRP10, RIP1, YLR342W-A, ATG33, and YOR238W, either showing an increased expression and/or overexpression or reduced expression and/or inactivation, depending on the experimental conditions.

Item 17. The method according to Item 15 or 16, further introducing into said cell the fungal gene HDA2 and/or PDI1, showing an increased expression and/or overexpression.

Item 18. The method according to any one of Items 15 to 17, wherein said at least one fungal gene is integrated into the genome as an expression cassette and/or extrachromosomally expressed, preferably using a replicative expression vector.

Item 19. Use of a yeast or filamentous fungal cell according to any one of Items 1 to 10 for producing at least one secreted protein of interest.

In summary, the present invention in particular provides the following items.

Item 20. A cell of Saccharomyces cerevisiae, producing at least one secreted protein of interest, wherein said cell comprises at least one fungal gene selected from the group consisting of ENO2, NMA2, PRY2, SUT074, TFG2, AVT2, TRM10, BNA7, and TOM22, wherein said at least one fungal gene shows increased expression and/or overexpression, and/or wherein said cell comprises at least one fungal gene selected from the group consisting of TLG2, MNT2, TPO2, ATG33, THR4, INP51, CUT901, YDR262W, MRP10, NDC1, and CMC1, wherein said at least one fungal gene shows reduced expression and/or inactivation, and optionally further comprising the fungal gene HDA2 and/or PDI1, showing an increased expression and/or overexpression.

Item 21. The yeast cell according to Item 20, wherein said cell comprises at least one fungal gene selected from the groups consisting of ENO2, NMA2, PRY2, SUT074, and TFG2, or AVT2, TRM10, PRY2, SUT074, BNA7, and TOM22, wherein said at least one fungal gene shows increased expression and/or overexpression, and/or wherein said cell comprises at least one fungal gene selected from the groups consisting of TLG2, CUT901, ATG33, THR4, YDR262W, and CMC1, or MRP10, TLG2, CUT901, ATG33, THR4, YDR262W, CMC1, MNT2, TPO2, and NDC1, preferably MNT2 and TPO2, wherein said at least one fungal gene shows reduced expression and/or inactivation, and optionally further comprising the fungal genes HDA2 and/or PDI1, showing an increased expression and/or overexpression, and/or INP51 showing an reduced expression and/or inactivation.

Item 23. The yeast cell according to Item 21 or 22, wherein said genes or SUTs or CUTs are furthermore selected from the group of genes or SUTs or CUTs having a value of log FC/FDR log FC/FDR of more than 40, preferably of more than 200, more preferred of more than 300, and most preferred of more than 500, based on the values as determined herein.

Item 24. The yeast cell according to any one of Items 21 to 23, wherein said yeast cell is from Saccharomyces cerevisiae strain ER.sec2.

Item 25. The yeast cell according to any one of Items 21 to 24, wherein said at least one secreted protein of interest also shows an increased expression and/or overexpression.

Item 26. The yeast cell according to any one of Items 21 to 25, wherein said at least one fungal gene showing increased expression and/or overexpression and/or showing reduced expression and/or inactivation is a native gene and/or is a recombinant gene, wherein preferably said recombinant gene is integrated into the genome as an expression cassette and/or extrachromosomally expressed, preferably using a replicative expression vector.

Item 27. The yeast cell according to any one of Items 21 to 26, wherein the cell furthermore comprises at least one additional recombinant secretion promoting gene, for example a gene for a chaperone, for a foldase and/or for a glycosylation-promoting protein.

Item 28. The yeast cell according to any one of Items 21 to 27, wherein the increased expression and/or overexpression or reduced expression and/or inactivation of the at least one fungal gene or the at least one additional recombinant secretion promoting gene is constitutive or inducible.

Item 29. The yeast cell according to any one of Items 21 to 28, wherein the cell produces the at least one secreted protein to about 30% or more, or about 40% or more, preferably about 50% or more, more preferably to about 75% or more, when compared to a control yeast or filamentous fungal cell.

Item 30. A method for producing a secreted protein in a yeast cell, comprising the steps of i) providing a cell of Saccharomyces cerevisiae producing at least one secreted protein of interest according to any one of Items 21 to 29, ii) culturing said yeast cell in suitable culture medium, and iii) isolating said secreted protein from said culture medium, and optionally further comprising suitably inducing the increased expression and/or overexpression or reduced expression and/or inactivation of the at least one fungal gene.

Item 31. The method according to Item 30, wherein preferably about 30% or more, or about 40% or more, preferably about 50% or more, more preferably to about 75% or more of said at least one secreted protein is produced, when compared to the production of a control yeast cell.

Item 32. A method for producing a yeast cell producing at least one secreted protein of interest, comprising introducing into said cell producing at least one secreted protein of interest at least one fungal gene selected from the group consisting of ENO2, NMA2, PRY2, SUT074, TFG2, AVT2, TRM10, BNA7, and TOM22, wherein said at least one fungal gene shows increased expression and/or overexpression, and/or wherein said cell comprises at least one fungal gene selected from the group consisting of TLG2, MNT2, TPO2, ATG33, THR4, INP51, CUT901, YDR262W, MRP10, NDC1, and CMC1, preferably MNT2, and TPO2, wherein said at least one fungal gene shows reduced expression and/or inactivation, and optionally further introducing into said cell a fungal gene selected from the group consisting of RIP1, YLR342W-A, and YOR238W, either showing an increased expression and/or overexpression or reduced expression and/or inactivation, depending on the experimental conditions, and/or optionally further introducing into said cell the fungal gene HDA2 and/or PDI1, showing an increased expression and/or overexpression.

Item 33. The method according to any one of Items 30 to 32, wherein said at least one fungal gene is integrated into the genome as an expression cassette and/or extrachromosomally expressed, preferably using a replicative expression vector.

Item 34. Use of a yeast cell according to any one of Items 21 to 29 for producing at least one secreted protein of interest.

The present invention will now be described further in the following examples with reference to the accompanying Figure, nevertheless, without being limited thereto. For the purposes of the present invention, all references as cited herein are incorporated by reference in their entireties.

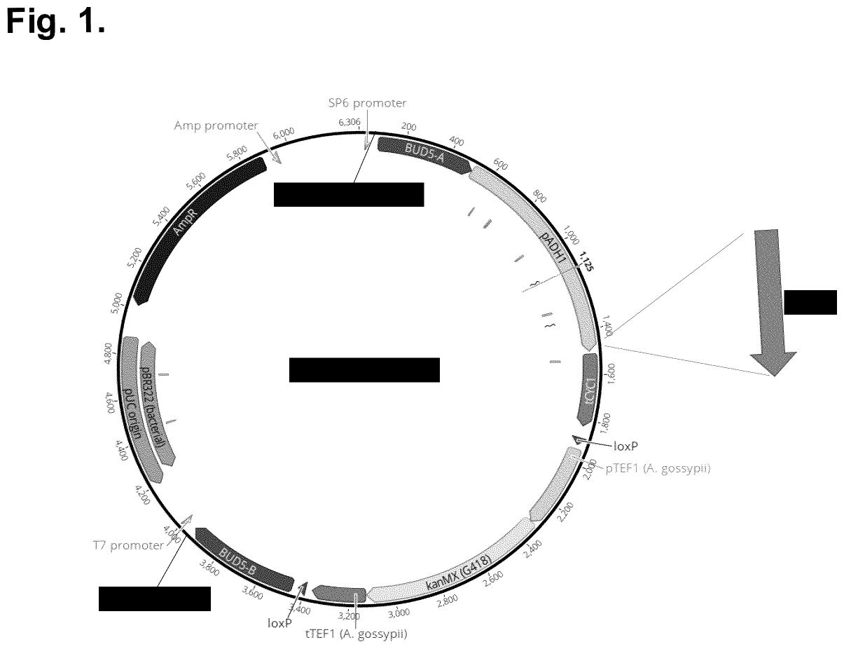

FIG. 1 shows the map of plasmid pLI410-062 as used in the methods according to the present invention.

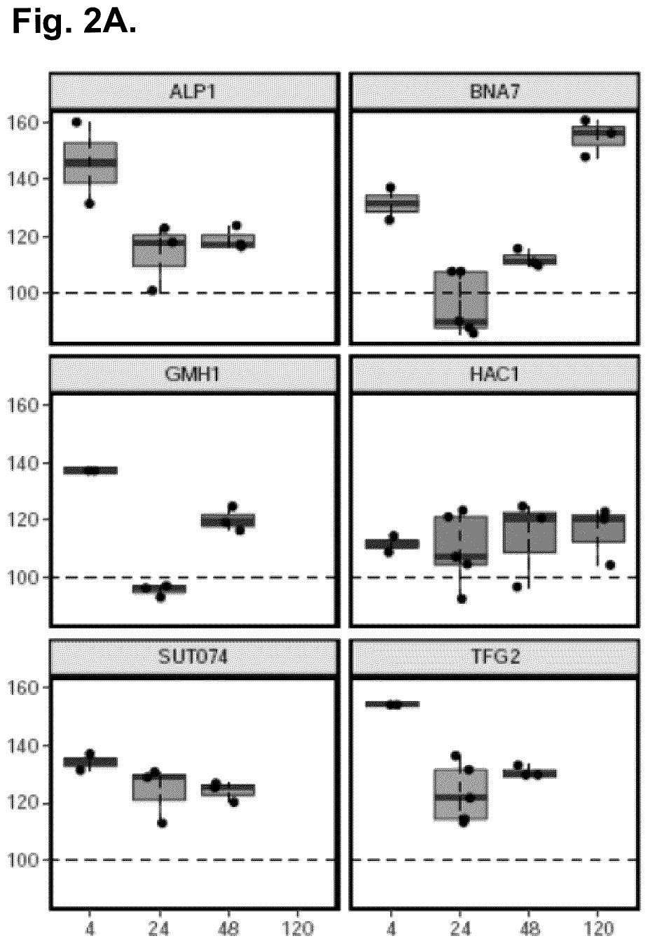

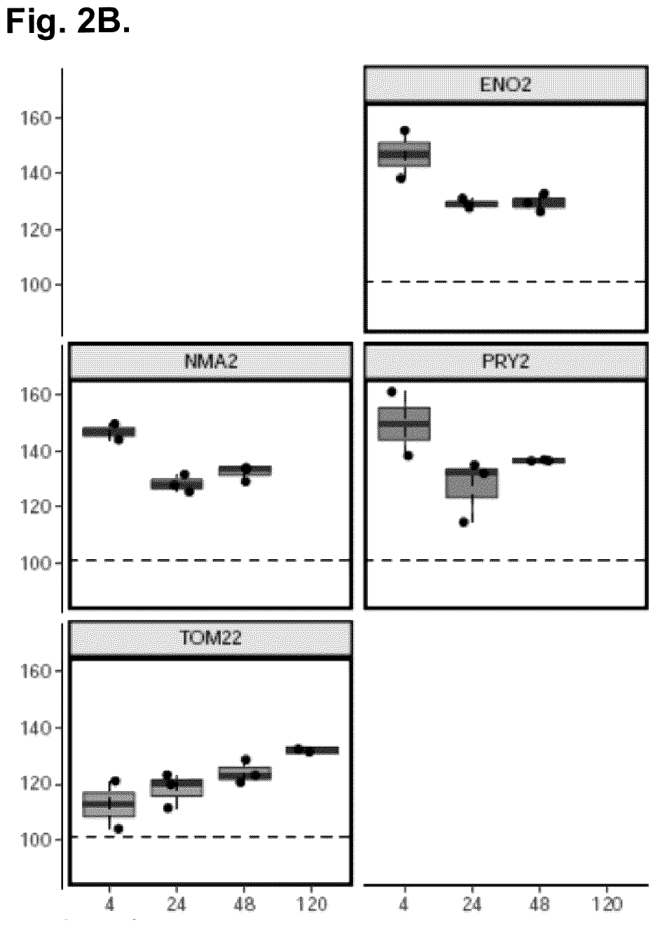

FIGS. 2 A and B shows the results of the α-amylase secretion measurements relative to baseline for selected genes of the present invention as box plots in % control over time (4, 24, 48, and 120 hours). Genes are ALP1, BNA7, GMH1, SUT074, TFG2, ENO2, NMA2, PRY2, and TOM22. HAC1 is control.

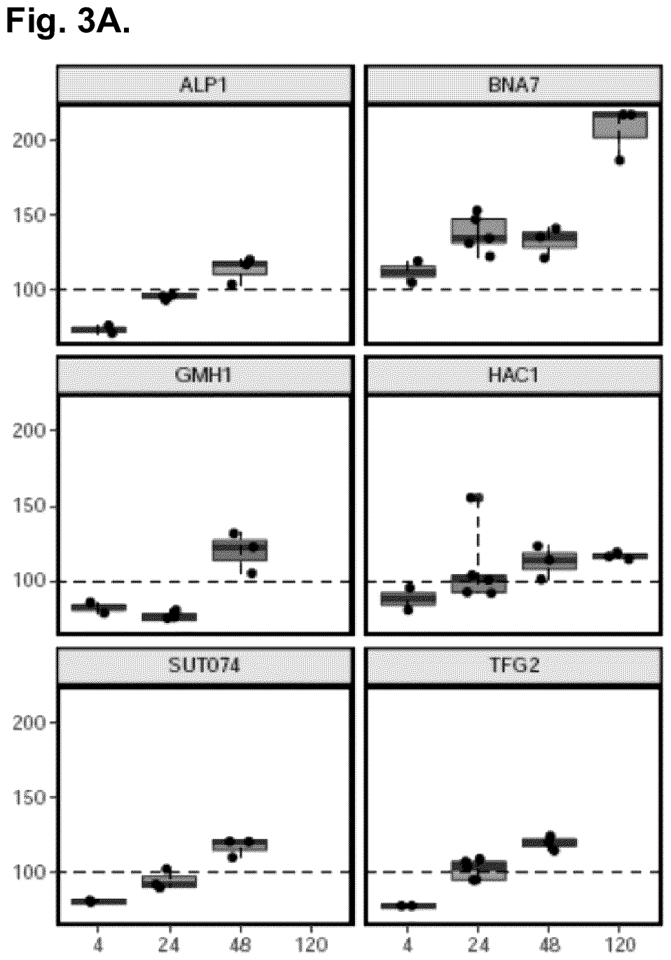

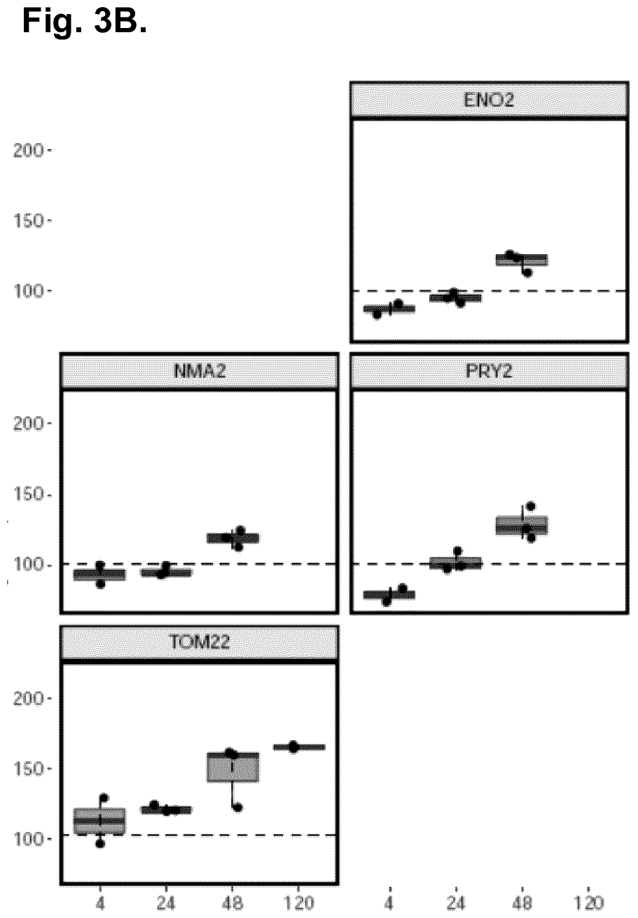

FIGS. 3 A and B shows the results of the α-amylase secretion measurements per cell for selected genes of the present invention as box plots in % control over time (4, 24, 48, and 120 hours). Genes are ALP1, BNA7, GMH1, SUT074, TFG2, ENO2, NMA2, PRY2, and TOM22. HAC1 is control.

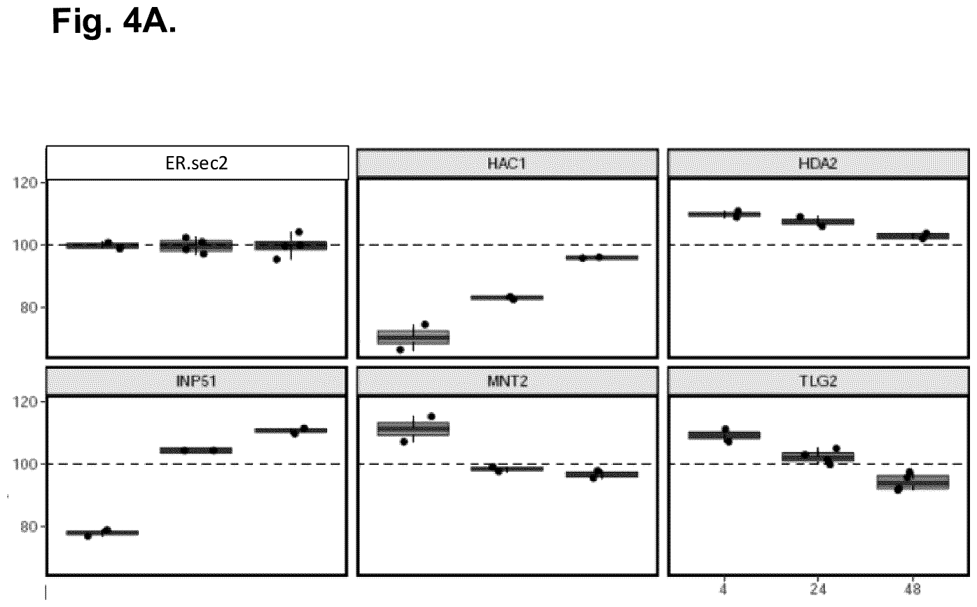

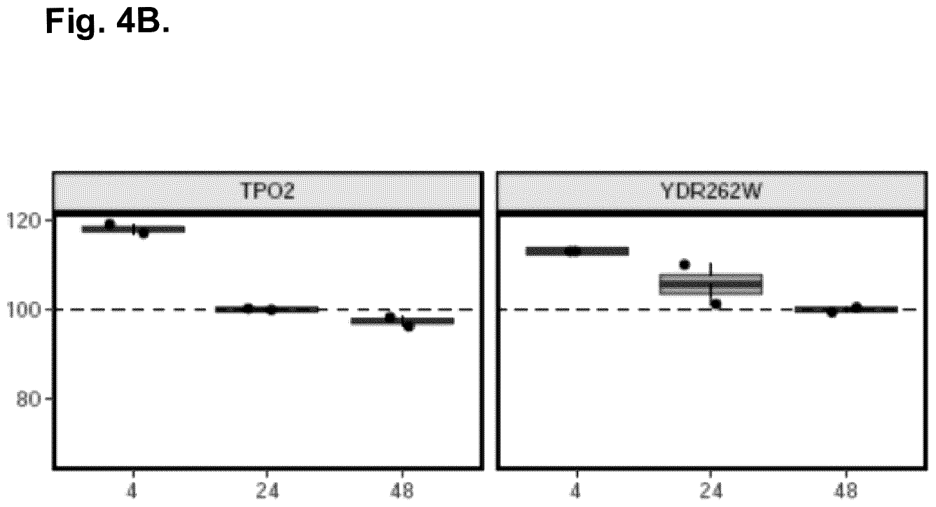

FIGS. 4 A and B shows the results of the α-amylase secretion measurements (total amylase) for selected genes of the present invention as box plots in % control over time (4, 24, and 48 hours). Genes are INP51, MNT2, TLG2, TPO2, and YDR262W. HAC1, HDA2 and ER.sec2 are controls.

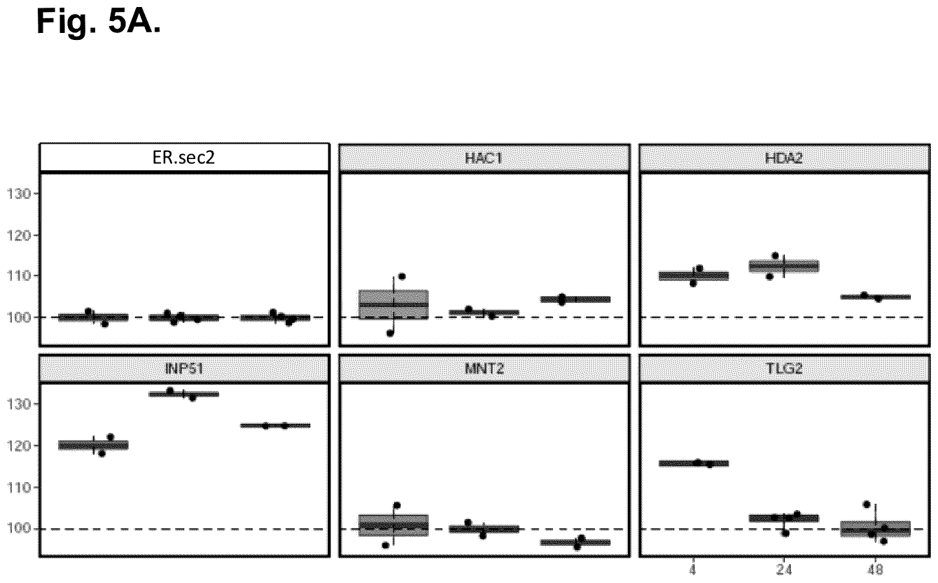

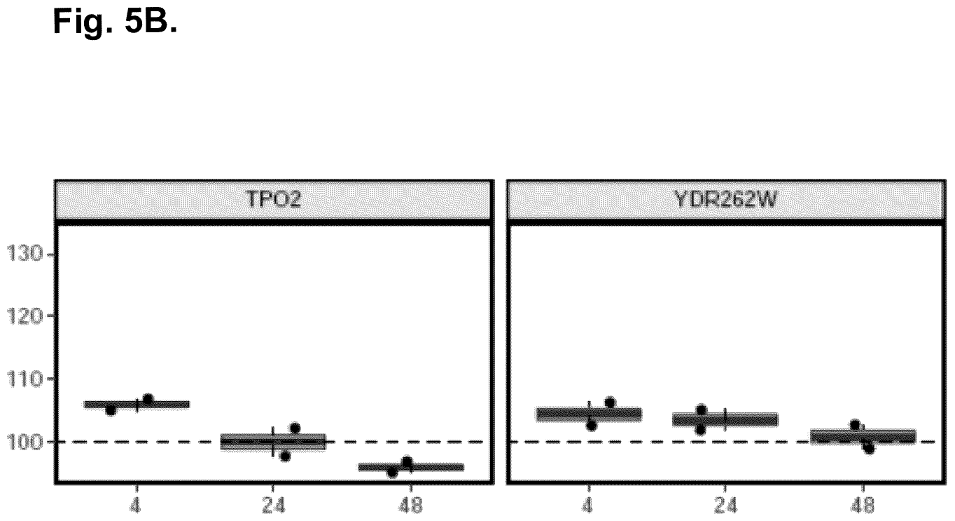

FIGS. 5 A and B shows the results of the α-amylase secretion measurements per cell for selected genes of the present invention as box plots in % control over time (4, 24, and 48 hours). Genes are INP51, MNT2, TLG2, TPO2, and YDR262W. HAC1, HDA2 and ER.sec2 are controls.

### EXAMPLES

### Materials and Methods

### Selection of Guide-RNA and Oligo Design

Guide RNA covering all known genes, SUTs, CUTs (for simplicity referred to as genes from here on) in S. cerevisiae were selected using Azimuth (Listgarten, J. et al. Prediction of off-target activities for the end-to-end design of CRISPR guide RNAs. Nat Biomed Eng2, 38-47 (2018).) and chosen to be as evenly distributed as possible in 5 bins of 100 bp each from 400 bp upstream to 100 bp downstream of the predicted transcription start site (TSS). This resulted in a library of 40890 guides for an average of approximately six guides per feature. The potential for off-target effects was minimized by blasting the individual guide RNAs (gRNA) against each other guide and all potential gRNA binding sites (4.7 M in total) throughout the genome and removing any guide with less than three mismatches. Oligos were ordered from Agilent using a design that optimizes the number of guides per oligo, each 190 bp oligo contains four individual 20 bp guide-RNA sequences interspersed with spacer sequences containing double Type II-S recognition sites, enabling restriction digest and release using BspQI with subsequent removal of the recognition site.

### Construction of Yeast Overexpression Strains

For overexpression of target genes using genome integration, candidate genes were cloned into plasmid pLI410-062 between the AscI and SbfI restriction sites, which was then linearized by NotI enzyme, and transformed into yeast strain ER.sec2. The plasmid integrates into the yeast chromosome at the BUDS locus (FIG. 1). For plasmid based overexpression of target genes, native candidate genes were cloned into plasmid p427-TEF between SpeI and SalI and transformed into yeast strain ER.sec2.

### Construction of Yeast Deletion Strains

Deletion strains were constructed by golden gate assembly of annealed oligos with gRNA sequences targeting the start and end position of the target gene, into sgRNA expression vector pWS082. The assembled plasmid and Cas9 expression vector pWS173 were linearized using EcoRV or BsmBI and co-transformed with annealed repair fragments, consisting of the joined 60 bp flanking regions of each target gene, which upon successful homology directed repair, resulted in the deletion of the target gene in ER.sec2.

The industrial Ethanol Red® (ER) yeast strain overexpressing an α-amylase (Amy6 from A. niger) was used as a model for the present invention. The person of skill in the art will be able to adapt the principles of the present invention to other fungal/yeast strains as shall be used, and—if required—to select suitable genes from the lists as disclosed in order to achieve the changes in expression(s) as disclosed herein.

### α-Amylase Activity Measurement

Preculture of YPD (Yeast extract Peptone Dextrose) was performed, either with 22h of culture on SD-2×SCAA, or 22 h and 96 h of culture on YPD. SD-2×SCAA medium was prepared as described previously (Hackel et al. 2006; Tyo et al. 2012), and the composition of SD-2×SCAA was as follows: 10 g/L glucose, 6.7 g/L yeast nitrogen base without amino acids, 2 g/L, KH2P04 (pH 6.0 by NaOH), and 1 g/L BSA, containing filter sterilized SCAA solution (190 mg/L arginine, 108 mg/L methionine, 52 mg/L tyrosine, 290 mg/L isoleucine, 440 mg/L lysine, 200 mg/L phenylalanine, 1,260 mg/L, glutamic acid, 400 mg/L aspartic acid, 380 mg/L valine, 220 mg/L threonine, 130 mg/L glycine, 400 mg/L leucine, 40 mg/L tryptophan, and 140 mg/L histidine) (see Liu et al., 2013—Correlation of cell growth and heterologous protein production by Saccharomyces cerevisiae).

The initial OD_{600 nm} was 0.1, and flasks of 250 ml+50 ml of medium were used. Culture density was measured at OD_{600 nm}.

For the assay, 100 μL of supernatant+900 μL of acetate buffer 50 mM pH5.5 were combined, and 10 μL of sample were incubated for 5 min at 40° C. in a PCR well plate.

Afterwards, 10 μL of BPNPG7 substrate was added, followed by incubation for 10 min at 40° C. The reaction was stopped by adding 150 μL of Trizma base 1%, followed by vortexing. The result was read at an OD of 400 nm, which generally required a prior step of 10 or 20-fold dilution.

### Calculation of α-Amylase Activity

The activity U was calculated as U=(ΔE₄₀₀/10)×(0.17/0.01)×(1/18.1)×D

E₄₀₀: Sample absorbance—blank absorbance, 10: time of reaction, 0.17: total volume of reaction, 0.01: volume of sample, 18.1: E_{mM} p-nitrophenol in Trizma base 1%, D: Dilution of sample. Normalization of α-amylase activity was performed with respective OD₆₀₀ nm.

### Results

Previous studies using microfluidics platforms, which screened for strains with an increased ability to secrete protein, using yeast cells treated with a mutagen, and encapsulated in a droplet with a suitable substrate, identified several strains that overexpressed α-amylase compared to the wild-type strain. These screens efficiently identified over-secretion strains by screening and sorting for increased protein secretion, but were to some degree hampered by the lack of a direct read-out of the affected genes, which necessitated whole-genome sequencing to identify the affected locus or loci.

The inventors utilized CRISPR with nuclease-null dCas9 to perturb a single gene per cell in a pooled format across the genome, coupled with microfluidic sorting of high fluorescence droplets using the same α-amylase assay described in the previous studies (Sjostrom, S. L. et al. High-throughput screening for industrial enzyme production hosts by droplet microfluidics. Lab Chip14, 806-813 (2013), Huang, M. et al. Microfluidic screening and whole-genome sequencing identifies mutations associated with improved protein secretion by yeast. Proc National Acad Sci112, E4689-E4696 (2015)), and a previously established chip design (Chaipan, C. et al. Single-Virus Droplet Microfluidics for High-Throughput Screening of Neutralizing Epitopes on HIV Particles. Cell Chem Biol24, 751-757.e3 (2017)); the guide RNA in this design also serves as a barcode, which allowed to directly identify genes for which an increase or decrease in expression is beneficial for improved protein secretion. As the background strain, a commercially available strain (Ethanol Red) was used, commonly used to produce bioethanol. The strain was engineered to express α-amylase by insertion of an expression cassette containing the codon-optimized α-amylase gene from (Aspergillus niger) in the HO-locus and then transformed with plasmid activation or repression libraries. The microfluidic system was used to create droplets containing cells from the transformed protein secreting strain, together with the fluorescent substrate, growth medium and a Tc to induce expression of the guide RNA, these droplets were incubated off chip, before sorting, with gating using thresholds adjusted to capture droplets of average size with the 2-5% highest fluorescence signal into a high fluorescence fraction with the remaining droplets passed passively into a low fluorescence fraction. Sequencing of the plasmid guide region from the sorted cells allowed to identify the guide population in each fraction.

Sequencing of the original assembled and transformed libraries identified a surviving gRNA representation of 72 and 86 percent, respectively, for the activation and the repression libraries following assembly, and 49 and 69 percent following re-transformation into yeast.

The activation screen identified 71 SUTs or CUTs as significantly enriched, SUTs generate stable transcripts that are thought to interact with other transcripts in both the nucleus and the cytosol, while CUTs are more unstable and quickly degraded upon transcription. An enrichment analysis of genes in the local genomic environment (1 kb interval centered on the SUT or CUT guide) identified genes from vacuolar, endosomal, and Golgi and related cellular components as the five most overrepresented cellular components within the range.

### Validation of Identified Genes

A set of genes identified as enriched, were selected for follow-up experimental validation of amylase over-secretion. Genes identified from the activation screens were validated via plasmid-based overexpression of the native gene, while genes from repression screens were validated via gene deletion in both alleles. The units of secreted α-amylase and cell density (OD 600) were measured at several time points after 4, 24, and 48 hours of growth. Overexpression of ENO2, NMA2, PRY2, SUT074 and TFG2 resulted in 20-40% increases in total α-amylase secretion after 24 and 48 hours, with even higher increases (35-60%) in the exponential phase after 4 hours of growth, while for BNA7 and TOM2 the relative amount of secreted protein per cell was instead significantly increased after 24 and 48 hours and 48 hours of growth respectively. Gene deletions of a smaller set of genes, resulted in increased total protein secretion for HDA2 (included as a positive control) MNT2, TPO2 after 4 hours, for INP51 protein secretion was initially significantly decreased after 4 hours, but increased over time and resulted in a significant increase after 48 hours. Deletion of INP51 also resulted in a significant increase in the secreted protein per cell during all measurements, while for HDA2 the increase was only significant for the first 24 hours.

The following genes and SUTs (stable uncharacterized transcripts) or CUTs (cryptic unstable transcripts) were identified as being of relevance, and relevance was defined as at least 2% increase of amylase activity (see above). See also FIG. 2.

1. Genes that were activated/overexpressed (integration of overexpression cassette into the genome and/or overexpression through a replicative plasmid) after statistical and enrichment analysis—preferred selection. log FC (log fold change) indicates the measure of enrichment, a higher value, equals a higher enrichment in the experiments as performed. FDR (false discovery rate) indicates the corrected p-value, a lower value means less variance between replicates as performed.

| Gene (common | | | |
|---|---|---|---|
| name, | | | |
| SUT or CUT or | | | |
| systematic | | | |
| designation) | Name and function (if known) | logFC | FDR |
| MIC19 | Component of the MICOS complex | 13.883 | 0.036 |
| TOM22 | Translocase of the Outer Mitochondrial membrane; | 13.781 | 0.008 |
| | responsible for initial import of mitochondrially | | |
| | directed proteins | | |
| NKP1 | Non-essential Kinetochore Protein | 13.389 | 0.012 |
| DML1 | Drosophila melanogaster Misato-Like protein, | 13.307 | 0.014 |
| | Essential protein involved in mtDNA inheritance | | |
| CUT859 | SUT or CUT | 13.152 | 0.033 |
| GAL80 | GALactose metabolism, Transcriptional regulator | 12.170 | 0.008 |
| | involved in the repression of GAL genes | | |
| APM3 | clathrin Adaptor Protein complex Medium chain | 12.088 | 0.020 |
| COQ10 | COenzyme Q, Coenzyme Q (ubiquinone) binding | 12.048 | 0.025 |
| | protein | | |
| BLM10 | BLeoMycin resistance, Proteasome activator | 12.008 | 0.030 |
| MDH1 | Malate DeHydrogenase, Mitochondrial malate | 11.915 | 0.008 |
| | dehydrogenase | | |
| VHS2 | Viable in a Hal3 Sit4 background, Regulator of septin | 11.838 | 0.032 |
| | dynamics | | |
| ASA1 | AStra Associated protein, Subunit of the ASTRA | 11.801 | 0.015 |
| | complex | | |
| TRP4 | TRYPtophan, Anthranilate phosphoribosyl | 11.698 | 0.019 |
| | transferase | | |
| YPS7 | YaPSin, Putative GPI-anchored aspartic protease | 11.620 | 0.030 |
| CUT824 | SUT or CUT | 11.529 | 0.041 |
| YOR318C | Gene of unknown function | 11.515 | 0.013 |
| PRM7 | Pheromone-Regulated Membrane protein | 11.485 | 0.023 |
| ERV46 | ER Vesicle, Protein localized to COPII-coated | 11.350 | 0.010 |
| | vesicles | | |
| FIT2 | Facilitator of Iron Transport, Mannoprotein that is | 11.287 | 0.034 |
| | incorporated into the cell wall | | |
| GPM3 | Glycerate PhosphoMutase | 11.062 | 0.019 |
| CUT892 | SUT or CUT | 10.972 | 0.050 |
| SRN2 | Suppressor of Rna mutations, Number 2 | 10.938 | 0.021 |
| SUT643 | SUT or CUT | 10.910 | 0.039 |
| CUT461 | SUT or CUT | 10.901 | 0.042 |
| THR4 | THReonine requiring, Threonine synthase | 10.840 | 0.047 |
| GMH1 | Gea1-6 Membrane-associated High-copy suppressor; | 10.780 | 0.055 |
| | Golgi membrane protein of unknown function | | |
| SOL1 | Suppressor Of Los1-1, Protein with a possible role in | 10.725 | 0.026 |
| | tRNA export | | |
| NAB6 | Nucleic Acid Binding protein, Putative RNA-binding | 10.674 | 0.013 |
| | protein | | |
| YPR148C | Gene of unknown function | 10.614 | 0.027 |
| ALP1 | Arginine transporter | 10.598 | 0.046 |
| CUT097 | SUT or CUT | 10.597 | 0.046 |
| ATG33 | AuTophaGy related, Mitochondrial mitophagy- | 10.585 | 0.030 |
| | specific protein | | |
| YOR316C-A | Gene of unknown function | 10.547 | 0.025 |
| SOG2 | Key component of the RAM signaling network; | 10.546 | 0.039 |
| | required for proper cell morphogenesis and cell | | |
| | separation after mitosis | | |
| MCM6 | MiniChromosome Maintenance, Protein involved in | 10.531 | 0.019 |
| | DNA replication | | |
| SUT230 | SUT or CUT | 10.507 | 0.010 |
| SUT419 | SUT or CUT | 10.398 | 0.027 |
| TIF11 | Translation Initiation Factor | 10.334 | 0.024 |
| TAF5 | TATA binding protein-Associated Factor, involved | 10.328 | 0.027 |
| | in RNA polymerase II transcription initiation and in | | |
| | chromatin modification | | |
| PHO91 | PHOsphate metabolism, Low-affinity vacuolar | 10.303 | 0.024 |
| | phosphate transporter | | |
| AIM32 | Altered Inheritance rate of Mitochondria, 2Fe—2S | 10.271 | 0.042 |
| | mitochondrial protein involved in redox quality | | |
| | control | | |
| ENO2 | ENOlase, Enolase II, a phosphopyruvate hydratase | 10.260 | 0.050 |
| UBA2 | UBiquitin Activating, Subunit of heterodimeric | 10.215 | 0.030 |
| | nuclear SUMO activating enzyme E1 with Aos1p | | |
| PUS5 | PseudoUridine Synthase | 10.197 | 0.030 |
| ERG1 | ERGosterol biosynthesis, Squalene epoxidase | 10.139 | 0.013 |
| SUT311 | SUT or CUT | 10.130 | 0.012 |
| KSS1 | Kinase Suppressor of Sst2 mutations, Mitogen- | 10.116 | 0.039 |
| | activated protein kinase (MAPK) | | |
| MRP10 | Mitochondrial Ribosomal Protein, Mitochondrial | 10.099 | 0.023 |
| | ribosomal protein of the small subunit | | |
| CUT598 | SUT or CUT | 10.099 | 0.046 |
| CUT188 | SUT or CUT | 10.073 | 0.026 |
| YOR238W | Gene of unknown function | 10.023 | 0.025 |
| EMW1 | Essential for Maintenance of the cell Wall, Essential | 15.549 | 0.071 |
| | conserved protein with a role in cell wall integrity | | |
| BNA7 | Biosynthesis of NAD, Formylkynurenine | 14.863 | 0.071 |
| | formamidase | | |
| SNR63 | Small Nucleolar RNA, C/D box small nucleolar | 14.717 | 0.071 |
| | RNA (snoRNA) | | |
| CCT3 | Chaperonin Containing TCP-1, Subunit of the | 14.647 | 0.071 |
| | cytosolic chaperonin Cct ring complex | | |
| PRY2 | Pathogen Related in Yeast, Sterol binding protein | 14.548 | 0.071 |
| | involved in the export of acetylated sterols | | |
| MAL11 | MALtose fermentation, High-affinity maltose | 14.484 | 0.071 |
| | transporter (alpha-glucoside transporter) | | |
| KRS1 | Lysyl (K) tRNA Synthetase | 14.290 | 0.072 |
| RAI1 | Rat1p Interacting Protein, Nuclear decapping | 14.254 | 0.071 |
| | endonuclease | | |
| SUT784 | SUT or CUT | 13.682 | 0.071 |
| YPR148C | Gene of unknown function | 13.572 | 0.071 |
| YEL1 | Yeast EFA6-Like, Guanine nucleotide exchange | 13.417 | 0.096 |
| | factor specific for Arf3p | | |
| CUT832 | SUT or CUT | 13.118 | 0.071 |
| NMA2 | Nicotinamide Mononucleotide Adenylyltransferase | 13.116 | 0.071 |
| VPS27 | Vacuolar Protein Sorting, Endosomal protein that | 12.963 | 0.071 |
| | forms a complex with Hse1p | | |
| SUT428 | SUT or CUT | 12.841 | 0.089 |
| PEX29 | PEroXisome related, ER-resident protein involved in | 12.477 | 0.071 |
| | peroxisomal biogenesis | | |
| YLR446W | Gene of unknown function | 12.369 | 0.071 |
| WBP1 | Wheat germ agglutinin-Binding Protein, Beta subunit | 12.078 | 0.087 |
| | of the oligosaccharyl transferase glycoprotein | | |
| | complex | | |
| AVT2 | Amino acid Vacuolar Transport, Putative transporter | 10.965 | 0.071 |
| CUT854 | SUT or CUT | 10.873 | 0.093 |
| TRM10 | Transfer RNA Methyltransferase, methylates the N-1 | 10.442 | 0.099 |
| | position of guanine at position 9 in tRNAs | | |
| SLX9 | Protein required for pre-rRNA processing | 9.996 | 0.012 |
| YPL077C | Gene of unknown function | 9.994 | 0.038 |
| PET122 | PETite colonies, Mitochondrial translational | 9.982 | 0.039 |
| | activator specific for the COX3 mRNA | | |
| TFG2 | Transcription Factor G; involved in both transcription | 9.973 | 0.090 |
| | initiation and elongation of RNA polymerase II | | |
| PUN1 | Plasma membrane protein Upregulated during | 9.950 | 0.027 |
| | Nitrogen stress | | |
| CUT152 | SUT or CUT | 9.936 | 0.020 |
| AIR2 | Arginine methyltransferase-Interacting RING finger | 9.886 | 0.044 |
| | protein, involved in nuclear RNA processing and | | |
| | degradation | | |
| CUT571 | SUT or CUT | 9.799 | 0.033 |
| RPS26B | Protein component of the small (40S) ribosomal | 9.789 | 0.023 |
| | subunit | | |
| RRT6 | Regulator of rDNA Transcription | 9.749 | 0.012 |
| RPC19 | RNA Polymerase C, RNA polymerase subunit AC19 | 9.715 | 0.047 |
| URA3 | URAcil requiring, Orotidine-5′-phosphate (OMP) | 9.687 | 0.046 |
| | decarboxylase | | |
| YGR045C | Gene of unknown function | 9.679 | 0.039 |
| SMC3 | Stability of MiniChromosomes, Subunit of the | 9.669 | 0.025 |
| | multiprotein cohesin complex | | |
| PNG1 | Peptide N-Glycanase | 9.654 | 0.019 |
| THI6 | THIamine biosynthesis, Thiamine-phosphate | 9.653 | 0.033 |
| | diphosphorylase and hydroxyethylthiazole kinase | | |
| MEU1 | Multicopy Enhancer of UAS2, Methylthioadenosine | 9.558 | 0.031 |
| | phosphorylase (MTAP) | | |
| CUT239 | SUT or CUT | 9.531 | 0.032 |
| NSE4 | Non-SMC Element, Component of the SMC5-SMC6 | 9.502 | 0.023 |
| | complex | | |
| SUT074 | SUT or CUT | 9.478 | 0.019 |
| AAH1 | Adenine AminoHydrolase, Adenine deaminase | 9.454 | 0.044 |
| | (adenine aminohydrolase) | | |
| RMD5 | Required for Meiotic nuclear Division, Component of | 9.452 | 0.024 |
| | GID Complex that confers ubiquitin ligase (U3) | | |
| | activity | | |
| CUT607 | SUT or CUT | 9.313 | 0.020 |
| ACS1 | Acetyl COA Synthetase, Acetyl-coA synthetase | 9.305 | 0.036 |
| | isoform | | |
| MNN1 | MaNNosyltransferase, Alpha-1,3- | 9.265 | 0.019 |
| | mannosyltransferase | | |
| ARH1 | Adrenodoxin Reductase Homolog, Oxidoreductase | 9.244 | 0.039 |
| | of the mitochondrial inner membrane | | |
| YHR140W | Gene of unknown function | 9.220 | 0.021 |
| CET1 | Capping Enzyme Triphosphatase, RNA 5′- | 9.203 | 0.019 |
| | triphosphatase involved in mRNA 5′ capping | | |
| RRB1 | Regulator of Ribosome Biogenesis, Specific | 9.185 | 0.030 |
| | assembly chaperone for ribosomal protein Rpl3p | | |
| YLR342W-A | Gene of unknown function | 9.166 | 0.010 |
| RPS22B | Ribosomal Protein of the Small subunit, Protein | 9.154 | 0.024 |
| | component of the small (40S) ribosomal subunit | | |
| CHS5 | CHitin Synthase-related, Component of the exomer | 9.143 | 0.027 |
| | complex | | |
| YIL165C | Gene of unknown function | 9.140 | 0.040 |
| SUT093 | SUT or CUT | 9.139 | 0.030 |
| LPX1 | Lipase of PeroXisomes, Peroxisomal matrix- | 9.114 | 0.039 |
| | localized lipase | | |
| NCA3 | Nuclear Control of ATPase, Protein involved in | 9.078 | 0.026 |
| | mitochondrion organization | | |
| EFG1 | Exit From G1, Ribosome biogenesis factor required | 9.063 | 0.040 |
| | for maturation of 18S rRNA | | |
| NBP35 | Nucleotide Binding Protein, Essential cytoplasmic | 9.055 | 0.042 |
| | iron-sulfur cluster binding protein | | |
| CUT765 | SUT or CUT | 9.038 | 0.037 |
| MSL1 | MUD Synthetic Lethal | 9.019 | 0.015 |
| SCD6 | Suppressor of Clathrin Deficiency, Repressor of | 9.004 | 0.025 |
| | translation initiation | | |
| ATG42 | AuTophaGy, Vacuolar serine-type carboxypeptidase | 9.001 | 0.028 |
| CHS6 | CHitin Synthase-related, Member of the ChAPs | 8.974 | 0.020 |
| | (Chs5p-Arf1p-binding proteins) family | | |
| COQ2 | COenzyme Q, Para hydroxybenzoate polyprenyl | 8.973 | 0.045 |
| | transferase | | |
| RPO31 | RNA Polymerase, RNA polymerase III largest | 8.969 | 0.044 |
| | subunit C160 | | |
| MKK1 | Mitogen-activated protein Kinase-Kinase, MAPKK | 8.958 | 0.030 |
| | involved in the protein kinase C signaling pathway | | |
| HED1 | High copy suppressor of rED1, Meiosis-specific | 8.903 | 0.025 |
| | protein | | |
| PBP2 | Pbp1p Binding Protein, RNA binding protein; has | 8.891 | 0.027 |
| | similarity to mammalian heterogeneous nuclear RNP | | |
| | K protein | | |
| BET5 | Blocked Early in Transport, Core component of | 8.890 | 0.019 |
| | transport protein particle (TRAPP) complexes I-III | | |
| CUT678 | SUT or CUT | 8.876 | 0.045 |
| YGR021W | Gene of unknown function | 8.823 | 0.012 |
| SUT474 | SUT or CUT | 8.811 | 0.042 |
| YGL159W | Gene of unknown function | 8.802 | 0.014 |
| IRC21 | Increased Recombination Centers, unknown function | 8.795 | 0.027 |
| VHR1 | VHt1 Regulator, Transcriptional activator | 8.760 | 0.046 |
| SPP1 | Set1c, Phd finger Protein, Subunit of COMPASS | 8.721 | 0.025 |
| | (Set1C) | | |
| PRP43 | Pre-mRNA Processing, RNA helicase in the DEAH- | 8.707 | 0.042 |
| | box family | | |
| ZRT1 | Zinc-Regulated Transporter, High-affinity zinc | 8.705 | 0.039 |
| | transporter of the plasma membrane; responsible for | | |
| | the majority of zinc uptake | | |
| YLR041W | Gene of unknown function | 8.687 | 0.044 |
| SUT711 | SUT or CUT | 8.686 | 0.039 |
| COX18 | Cytochrome c OXidase, Protein required for | 8.685 | 0.046 |
| | membrane insertion of C-terminus of Cox2p | | |
| CBP6 | Cytochrome B Protein synthesis, Mitochondrial | 8.678 | 0.043 |
| | protein required for translation of the COB mRNA | | |
| SUT575 | SUT or CUT | 8.651 | 0.042 |
| CLG1 | Cyclin-Like Gene, Cyclin-like protein that interacts | 8.651 | 0.047 |
| | with Pho85p | | |
| CUT213 | SUT or CUT | 8.610 | 0.036 |
| QCR10 | ubiQuinol-cytochrome C oxidoReductase, Subunit of | 8.604 | 0.019 |
| | the ubiqunol-cytochrome c oxidoreductase complex | | |
| SNR3 | Small Nucleolar RNA, H/ACA box small nucleolar | 8.571 | 0.044 |
| | RNA (snoRNA) | | |
| MSS2 | Mitochondrial Splicing, Peripherally bound inner | 8.559 | 0.023 |
| | membrane protein of the mitochondrial matrix | | |
| CUT505 | SUT or CUT | 8.557 | 0.039 |
| YOS1 | Yip One Suppressor, Integral membrane protein | 8.540 | 0.023 |
| | required for ER to Golgi transport | | |
| SUT073 | SUT or CUT | 8.519 | 0.033 |
| UTP21 | U Three Protein, Subunit of U3-containing 90S | 8.511 | 0.039 |
| | preribosome and SSU processome complexes | | |
| ACA1 | ATF/CREB Activator, ATF/CREB family basic | 8.478 | 0.045 |
| | leucine zipper (bZIP) transcription factor | | |
| CUT632 | SUT or CUT | 8.475 | 0.039 |
| RIP1 | Rieske Iron-sulfur Protein, Ubiquinol-cytochrome-c | 8.466 | 0.037 |
| | reductase | | |
| HUL5 | Hect Ubiquitin Ligase, Multiubiquitin chain | 8.383 | 0.042 |
| | assembly factor (E4) | | |
| CUT727 | SUT or CUT | 8.373 | 0.030 |
| RPL35B | Ribosomal 60S subunit protein L35B | 8.360 | 0.019 |
| CUT184 | SUT or CUT | 8.304 | 0.039 |
| CUT420 | SUT or CUT | 8.300 | 0.023 |
| YFL041W-A | Gene of unknown function | 8.290 | 0.010 |
| SUT460 | SUT or CUT | 8.248 | 0.023 |
| ATG10 | AuTophaGy related, Conserved E2-like conjugating | 8.244 | 0.019 |
| | enzyme | | |
| MFA1 | Mating Factor A, Mating pheromone a-factor | 8.231 | 0.023 |
| UGX2 | Protein of unknown function | 8.226 | 0.023 |
| TRK2 | TRansport of potassium (K), Component of the | 8.218 | 0.027 |
| | Trk1p-Trk2p potassium transport system | | |
| CUT704 | SUT or CUT | 8.201 | 0.041 |
| SUT083 | SUT or CUT | 8.189 | 0.025 |
| TRE1 | Transferrin REceptor like, Transferrin receptor-like | 8.183 | 0.046 |
| | protein | | |
| RVS161 | Reduced Viability on Starvation, Amphiphysin-like | 8.110 | 0.034 |
| | lipid raft protein | | |
| LEA1 | Looks Exceptionally like U2A, Component of U2 | 8.092 | 0.044 |
| | snRNP complex | | |
| EBP2 | EBNA1-binding protein (homolog), Required for 25S | 8.089 | 0.030 |
| | rRNA maturation and 60S ribosomal subunit | | |
| | assembly; | | |
| THI80 | THIamine metabolism, Thiamine pyrophosphokinase | 8.071 | 0.012 |
| CTI6 | Cyc8-Tup1 Interacting protein, Component of the | 8.065 | 0.019 |
| | Rpd3L histone deacetylase complex | | |
| CUT322 | SUT or CUT | 8.002 | 0.027 |
| XPT1 | Xanthine Phosphoribosyl Transferase, Xanthine- | 7.984 | 0.036 |
| | guanine phosphoribosyl transferase | | |
| MRPL35 | Mitochondrial Ribosomal Protein, Large subunit | 7.963 | 0.031 |
| YPL025° C. | Gene of unknown function | 7.962 | 0.037 |
| SUT737 | SUT or CUT | 7.950 | 0.025 |
| PGA2 | Processing of Gaslp and ALP, Essential protein | 7.941 | 0.046 |
| | required for maturation of Gas1p and Pho8p | | |
| ULP2 | UbL-specific Protease, Peptidase that deconjugates | 7.935 | 0.033 |
| | Smt3/SUMO-1 peptides from proteins | | |
| MRX16 | Mitochondrial oRganization of gene expression | 7.917 | 0.044 |
| | (MIOREX), Protein that associates with the large | | |
| | mitoribosomal subunit | | |
| EST1 | Ever Shorter Telomeres, TLC1 RNA-associated | 7.911 | 0.042 |
| | factor involved in telomere length regulation | | |
| NUP100 | NUclear Pore, FG-nucleoporin component of central | 7.902 | 0.021 |
| | core of the nuclear pore complex | | |
| IES3 | Ino Eighty Subunit, Subunit of the INO80 chromatin | 7.880 | 0.031 |
| | remodeling complex | | |
| ATG39 | AuTophaGy related, Autophagy receptor with a role | 7.876 | 0.038 |
| | in degradation of the ER and nucleus | | |
| YMR084W | Gene of unknown function | 7.850 | 0.027 |
| SUT428 | SUT or CUT | 7.827 | 0.030 |
| YPL119C-A | Gene of unknown function | 7.791 | 0.031 |
| MIN8 | mitochondrial MINi protein of 8 kDa | 7.783 | 0.027 |
| CUT490 | SUT or CUT | 7.779 | 0.045 |
| SUT287 | SUT or CUT | 7.708 | 0.027 |
| KEL3 | KELch | 7.705 | 0.027 |
| SUT678 | SUT or CUT | 7.699 | 0.025 |
| SEC3 | SECretory, Subunit of the exocyst complex | 7.691 | 0.045 |
| SOL4 | Suppressor Of Los1-1, 6-phosphogluconolactonase | 7.678 | 0.030 |
| SIS2 | SIt4 Suppressor, Negative regulatory subunit of | 7.650 | 0.026 |
| | protein phosphatase 1 (Ppz1p) | | |
| CUT915 | SUT or CUT | 7.649 | 0.044 |
| RRP3 | Ribosomal RNA Processing, Protein involved in | 7.635 | 0.034 |
| | rRNA processing | | |
| ESA1 | Catalytic subunit of the histone acetyltransferase | 7.612 | 0.031 |
| | complex (NuA4) | | |
| PCL8 | Pho85 CycLin, Cyclin | 7.581 | 0.046 |
| TRX3 | ThioRedoXin, Mitochondrial thioredoxin | 7.579 | 0.033 |
| YKL115C | Gene of unknown function | 7.530 | 0.043 |
| EMP65 | ER Membrane Protein of 65 kDa, Integral membrane | 7.520 | 0.029 |
| | protein of the ER | | |
| ZDS1 | Zillion Different Screens, Protein with a role in | 7.488 | 0.049 |
| | regulating Swe1p-dependent polarized growth | | |
| CUT167 | SUT or CUT | 7.486 | 0.016 |
| SOD1 | SuperOxide Dismutase, Cytosolic copper-zinc | 7.471 | 0.019 |
| | superoxide dismutase | | |
| UBR2 | Cytoplasmic ubiquitin-protein ligase (E3 | 7.470 | 0.044 |
| LSP1 | Long chain bases Stimulate Phosphorylation, | 7.391 | 0.031 |
| | Eisosome core component | | |
| SNR81 | H/ACA box small nucleolar RNA (snoRNA) | 7.389 | 0.030 |
| RGD3 | GTPase activating protein (GAP) for Rho3p | 7.370 | 0.032 |
| YTP1 | Yeast putative Transmembrane Protein, Probable | 7.365 | 0.042 |
| | type-III integral membrane protein of unknown | | |
| | function | | |
| SMY2 | Suppressor of MY02-66, involved in COPII vesicle | 7.352 | 0.034 |
| | formation | | |
| CUT449 | SUT or CUT | 7.340 | 0.024 |
| FIN1 | Filaments In between Nuclei, Spindle pole body- | 7.335 | 0.039 |
| | related intermediate filament protein | | |
| YKL106C-A | Gene of unknown function | 7.293 | 0.021 |
| YAR019W-A | Gene of unknown function | 7.280 | 0.019 |
| CCH1 | Calcium Channel Homolog, Voltage-gated high- | 7.270 | 0.031 |
| | affinity calcium channel | | |
| AYR1 | 1-AcyldihYdroxyacetone-phosphate Reductase, | 7.243 | 0.012 |
| | ifunctional triacylglycerol lipase and 1-acyl DHAP | | |
| | reductase | | |
| SUT573 | SUT or CUT | 7.234 | 0.042 |
| VNX1 | Vacuolar Na+/H+ eXchanger, Calcium/H+ antiporter | 7.232 | 0.010 |
| | localized to the endoplasmic reticulum membrane | | |
| FOL3 | FOLic acid synthesis, Dihydrofolate synthetase | 7.215 | 0.032 |
| SUT511 | SUT or CUT | 7.212 | 0.026 |
| GIS4 | GIg1-2 Suppressor, proposed to be involved in the | 7.196 | 0.027 |
| | RAS/cAMP signaling pathway | | |
| CUT743 | SUT or CUT | 7.171 | 0.034 |
| RPL24A | Ribosomal 60S subunit protein L24A | 7.169 | 0.039 |
| HMT1 | HnRNP MethylTransferase, Nuclear SAM- | 7.163 | 0.026 |
| | dependent mono- and asymmetric methyltransferase | | |
| SUT333 | SUT or CUT | 7.141 | 0.031 |
| SPP2 | Suppressor of PrP, Essential protein that promotes the | 7.137 | 0.027 |
| | first step of splicing | | |
| SUT128 | SUT or CUT | 7.120 | 0.049 |
| SMC6 | Structural Maintenance of Chromosomes, Subunit of | 7.120 | 0.047 |
| | the SMC5-SMC6 complex | | |
| PHR1 | PHotoreactivation Repair deficient, DNA photolyase | 7.119 | 0.030 |
| | involved in photoreactivation | | |
| RPS15 | Protein component of the small (40S) ribosomal | 7.072 | 0.012 |
| | subunit | | |
| CUT642 | SUT or CUT | 7.066 | 0.025 |
| GYP7 | Gtpase-activating protein for Ypt7 Protein, GTPase- | 7.063 | 0.021 |
| | activating protein for yeast Rab family members | | |
| tK(CUU)K | Lysine tRNA (tRNA-Lys) | 7.034 | 0.041 |
| CUT896 | SUT or CUT | 7.026 | 0.041 |
| SLM5 | Synthetic Lethal with Mss4, Mitochondrial | 7.024 | 0.039 |
| | asparaginyl-tRNA synthetase | | |
| CUT586 | SUT or CUT | 7.020 | 0.038 |
| CUT158 | SUT or CUT | 7.003 | 0.030 |
| RRP12 | Ribosomal RNA Processing, Protein required for | 7.002 | 0.031 |
| | export of the ribosomal subunits | | |
| CUT276 | SUT or CUT | 6.84 | 0.026 |
| CUT480 | SUT or CUT | 6.81 | 0.030 |
| SUT751 | SUT or CUT | 6.75 | 0.023 |
| SUT251 | SUT or CUT | 6.30 | 0.035 |
| CUT643 | SUT or CUT | 5.48 | 0.021 |

1a. Gene to be Preferably Combined with the Preferred Selection

| | | | |
|---|---|---|---|
| PDI1 | Protein Disulfide Isomerase | 12.524 | 0.072 |

2. Genes or SUTs or CUTs that were inactivated/repressed after statistical and enrichment analysis—preferred selection. log FC (log fold change) indicates the measure of enrichment, a higher value, equals a higher enrichment in the experiments as performed. FDR (false discovery rate) indicates the corrected p-value, a lower value means less variance between replicates as performed.

| Gene | | | |
|---|---|---|---|
| (common | | | |
| name, SUT | | | |
| or CUT or | | | |
| systematic | | | |
| designation) | Name and function (if known) | logFC | FDR |
| TLG2 | T-snare affecting a Late Golgi compartment, Syntaxin-like t- | 13.51 | 0.010 |
| | SNARE | | |
| CUT901 | SUT or CUT | 11.72 | 0.009 |
| ATG33 | AuTophaGy related, Mitochondrial mitophagy-specific protein | 11.53 | 0.009 |
| THR4 | THReonine requiring, Threonine synthase | 11.49 | 0.009 |
| YDR262W | Gene of unknown function | 10.92 | 0.009 |
| CMC1 | Cx9C Mitochondrial protein necessary for full assembly of | 10.86 | 0.009 |
| | Cytochrome c oxidase, Copper-binding protein of the | | |
| | mitochondrial intermembrane space | | |
| MRP17 | Mitochondrial ribosomal protein of the small subunit | 10.20 | 0.019 |
| YPT52 | Yeast Protein Two, Endosomal Rab family GTPase; required | 8.91 | 0.043 |
| | for vacuolar protein sorting | | |
| CUT312 | SUT or CUT | 8.90 | 0.014 |
| MRPS5 | Mitochondrial Ribosomal Protein, Small subunit | 8.87 | 0.022 |
| RDR1 | Repressor of Drug Resistance, Transcriptional repressor | 8.65 | 0.042 |
| | involved in regulating multidrug resistance | | |
| DAL7 | Degradation of Allantoin, Malate synthase | 8.55 | 0.009 |
| RPL20A | Ribosomal 60S subunit protein L20A | 8.19 | 0.025 |
| YBR137W | Gene of unknown function | 8.11 | 0.056 |
| RPL36B | Ribosomal 60S subunit protein L36B | 8.02 | 0.028 |
| YEL008C-A | Gene of unknown function | 7.85 | 0.062 |
| RAX1 | Revert to Axial, Protein involved in establishing bud site | 7.65 | 0.019 |
| | selection | | |
| INP51 | INositol polyphosphate 5-Phosphatase | 7.51 | 0.102 |
| CUT729 | SUT or CUT | 7.27 | 0.066 |
| UBP8 | UBiquitin-specific processing Protease, Ubiquitin-specific | 7.18 | 0.066 |
| | protease component of the SAGA acetylation complex | | |
| CUT258 | SUT or CUT | 7.10 | 0.089 |
| YLR342W- | Gene of unknown function | 7.09 | 0.025 |
| A | | | |
| SUT568 | SUT or CUT | 7.04 | 0.027 |
| PEX7 | PEroXin, Peroxisomal signal receptor for peroxisomal matrix | 7.00 | 0.024 |
| | proteins | | |
| MSD1 | Mitochondrial aminoacyl-tRNA Synthetase, Aspartate (D) | 6.97 | 0.089 |
| CUT136 | SUT or CUT | 6.88 | 0.039 |
| TIM10 | Translocase of the Inner Membrane, Essential protein of the | 6.84 | 0.064 |
| | mitochondrial intermembrane space | | |
| CUT361 | SUT or CUT | 6.83 | 0.037 |
| snR51 | Small Nucleolar RNA | 6.80 | 0.085 |
| TAL1 | TransALdolase, Transaldolase, enzyme in the non-oxidative | 6.74 | 0.069 |
| | pentose phosphate pathway | | |
| RIP1 | Rieske Iron-sulfur Protein, Ubiquinol-cytochrome-c reductase | 6.65 | 0.058 |
| MRP10 | Mitochondrial Ribosomal Protein, Mitochondrial ribosomal | 6.63 | 0.051 |
| | protein of the small subunit | | |
| SUT078 | SUT or CUT | 6.52 | 0.074 |
| MRP51 | Mitochondrial Ribosomal Protein, Mitochondrial ribosomal | 6.51 | 0.065 |
| | protein of the small subunit | | |
| GLO3 | GLyOxalase, ADP-ribosylation factor GTPase activating | 6.51 | 0.053 |
| | protein (ARF GAP); involved in ER-Golgi transport | | |
| EHD3 | 3-hydroxyisobutyryl-CoA hydrolase | 6.50 | 0.025 |
| HER1 | Hmg2p ER Remodeling, Protein of unknown function | 6.48 | 0.051 |
| NMA111 | Nuclear Mediator of Apoptosis, Serine protease and general | 6.45 | 0.041 |
| | molecular chaperone | | |
| PBP4 | Pbp1p binding protein | 6.28 | 0.044 |
| MFB1 | Mitochondria-associated F-box protein; involved in | 6.25 | 0.098 |
| | maintenance of normal mitochondrial morphology | | |
| IKI3 | Insensitive to KIller toxin, Subunit of Elongator complex | 6.21 | 0.031 |
| NDL1 | NuDeL homolog, Homolog of nuclear distribution factor NudE | 6.15 | 0.057 |
| SUT433 | SUT or CUT | 5.99 | 0.022 |
| YOR238W | Gene of unknown function | 5.91 | 0.054 |
| SUT750 | SUT or CUT | 5.86 | 0.016 |
| QDR2 | QuiniDine Resistance, Plasma membrane transporter of the | 5.84 | 0.020 |
| | major facilitator superfamily | | |
| RDI1 | Rho GDP Dissociation Inhibitor | 5.79 | 0.023 |
| SUT014 | SUT or CUT | 5.76 | 0.059 |
| CUT437 | SUT or CUT | 5.75 | 0.045 |
| MSC6 | Meiotic Sister-Chromatid recombination, Multicopy | 5.66 | 0.055 |
| | suppressor of HER2 involved in mitochondrial translation | | |
| SUT497 | SUT or CUT | 5.54 | 0.072 |
| YCR051W | Gene of unknown function | 5.52 | 0.076 |
| MRPL33 | Mitochondrial Ribosomal Protein, Large subunit | 5.47 | 0.024 |
| RPL14A | Ribosomal 60S subunit protein L14A | 5.46 | 0.077 |
| TRM7 | 2′-O-ribose methyltransferase | 5.43 | 0.081 |
| RNH202 | Ribonuclease H2 subunit; required for RNase H2 activity | 5.43 | 0.083 |
| RTC5 | Restriction of Telomere Capping, Protein of unknown function | 5.38 | 0.060 |
| SUT027 | SUT or CUT | 5.34 | 0.058 |
| CDC5 | Cell Division Cycle, Polo-like kinase essential for mitotic cell | 5.33 | 0.070 |
| | cycle | | |
| SUT729 | SUT or CUT | 5.30 | 0.076 |
| YOR131C | Gene of unknown function | 5.28 | 0.078 |
| CUT665 | SUT or CUT | 5.12 | 0.097 |
| GLG2 | Glycogenin-Like Gene, Glycogenin glucosyltransferase | 5.12 | 0.079 |
| SUT268 | SUT or CUT | 4.89 | 0.087 |
| SUT705 | SUT or CUT | 4.87 | 0.086 |
| MED4 | MEDiator complex, Subunit of the RNA polymerase II | 4.61 | 0.093 |
| | mediator complex | | |
| RCR2 | Resistance to Congo Red, Vacuolar ubiquitin ligase-substrate | 4.59 | 0.055 |
| | adaptor | | |
| EFB1 | Elongation Factor Beta, Translation elongation factor 1 beta | 4.58 | 0.036 |
| RXT2 | Component of the histone deacetylase Rpd3L complex | 4.49 | 0.073 |
| KGD1 | alpha-KetoGlutarate Dehydrogenase, Subunit of the | 4.42 | 0.093 |
| | mitochondrial alpha-ketoglutarate dehydrogenase complex | | |
| TUP1 | dTMP-UPtake, General repressor of transcription | 4.35 | 0.080 |
| RNH203 | Ribonuclease H2 subunit | 4.31 | 0.096 |
| YDR338C | Gene of unknown function | 3.92 | 0.029 |
| SED1 | Suppression of Exponential Defect, Major stress-induced | 3.81 | 0.089 |
| | structural GPI-cell wall glycoprotein | | |
| CUT522 | SUT or CUT | 3.75 | 0.092 |
| HIS2 | HIStidine requiring, Histidinolphosphatase | 3.74 | 0.090 |
| SUT145 | SUT or CUT | 3.67 | 0.072 |
| MET17 | METhionine requiring, O-acetyl homoserine-O-acetyl serine | 3.58 | 0.063 |
| | sulfhydrylase | | |
| APC4 | Anaphase Promoting, Subunit of the Anaphase-Promoting | 3.58 | 0.077 |
| | Complex/Cyclosome (APC/C) | | |
| NKP2 | Non-essential Kinetochore Protein, Central kinetochore protein | 3.54 | 0.022 |
| | and subunit of the Ctf19 complex | | |
| MKK2 | Mitogen-activated Kinase Kinase, MAPKK involved in the | 3.05 | 0.042 |
| | protein kinase C signaling pathway | | |
| NDC1 | Nuclear Division Cycle, Subunit of the transmembrane ring of | 14.18 | 0.079 |
| | the nuclear pore complex (NPC) | | |
| PET100 | PETite colonies, Chaperone that facilitates the assembly of | 12.69 | 0.086 |
| | cytochrome c oxidase | | |
| NIP7 | Nuclear ImPort, Nucleolar protein required for 60S ribosome | 12.54 | 0.086 |
| | subunit biogenesis | | |
| VHT1 | Vitamin H Transporter, High-affinity plasma membrane H+- | 12.31 | 0.086 |
| | biotin (vitamin H) symporter | | |
| SUT685 | SUT or CUT | 12.07 | 0.086 |
| BNI5 | Bud Neck Involved, Linker protein responsible for recruitment | 11.96 | 0.086 |
| | of myosin to the bud neck | | |
| SNA3 | Sensitivity to NA+, Protein involved in efficient MVB sorting | 11.93 | 0.086 |
| | of proteins to the vacuole | | |
| EGH1 | Cryptococcus neoformans EGCrP2 Homolog, Steryl-beta- | 11.81 | 0.086 |
| | glucosidase with broad specificity for aglycones | | |
| MRP4 | Mitochondrial ribosomal protein of the small subunit | 11.67 | 0.086 |
| POB3 | PO11 Binding, Subunit of the heterodimeric FACT complex | 10.87 | 0.086 |
| | (Spt16p-Pob3p) | | |
| PIB2 | PtdIns(3)p-Binding, Phosphatidylinositol 3-phosphate binding | 10.80 | 0.086 |
| | protein | | |
| SUT317 | SUT or CUT | 10.74 | 0.086 |
| NTO1 | NuA Three Orf, Subunit of the NuA3 histone acetyltransferase | 10.62 | 0.086 |
| | complex | | |
| YKL024C | URA6 Uridylate kinase; catalyzes the seventh enzymatic step | 7.08 | 0.102 |
| | in the de novo biosynthesis of pyrimidines | | |
| YGL116W | CDC20 Activator of anaphase-promoting complex/cyclosome | 6.44 | 0.140 |
| | (APC/C) | | |
| YLR118C | TML25 Acyl-protein thioesterase responsible for | 6.24 | 0.149 |
| | depalmitoylation of Gpalp | | |
| YFR031C-A | RPL2A Ribosomal 60S subunit protein L2A | 6.16 | 0.104 |
| YGL190C | CDC55 Regulatory subunit B of protein phosphatase 2A | 6.02 | 0.134 |
| | (PP2A) | | |
| YDL108W | KIN28 Ser/Thr protein kinase and subunit of TFIIK, a TFIIH | 5.81 | 0.101 |
| | subassembly | | |
| YMR128W | ECM16 Essential DEAH-box ATP-dependent RNA helicase | 5.57 | 0.118 |
| | specific to U3 snoRNP | | |
| YBR253W | SRB6 Subunit of the RNA polymerase II mediator complex | 5.33 | 0.142 |
| YJR113C | RSM7 Mitochondrial ribosomal protein of the small subunit | 5.28 | 0.134 |
| YIL031W | ULP2 Peptidase that deconjugates Smt3/SUMO-1 peptides | 5.20 | 0.140 |
| | from proteins | | |
| YGR109C | CLB6 B-type cyclin involved in DNA replication during S | 5.15 | 0.141 |
| | phase | | |
| YBR282W | MRPL27 Mitochondrial ribosomal protein of the large subunit | 5.14 | 0.136 |
| YMR125W | STO1 Large subunit of the nuclear mRNA cap-binding protein | 4.97 | 0.106 |
| | complex | | |
| YMR236W | TAF9 Subunit (17 kDa) of TFIID and SAGA complexes | 4.96 | 0.125 |
| YDR411C | DFM1 Endoplasmic reticulum (ER) localized protein | 4.71 | 0.108 |
| YML029W | USA1 Scaffold subunit of the Hrdlp ubiquitin ligase | 4.55 | 0.106 |
| YDL033C | SLM3 tRNA-specific 2-thiouridylase | 4.50 | 0.131 |
| YPL050C | MNN9 Subunit of Golgi mannosyltransferase complex | 4.42 | 0.102 |
| YHR171W | ATG7 Autophagy-related protein and dual specificity member | 4.32 | 0.143 |
| | of the E1 family | | |
| YDR352W | YPQ2 Putative vacuolar membrane transporter for cationic | 4.27 | 0.137 |
| | amino acids | | |

3. Genes that were either overexpressed or inactivated/repressed depending on experimental conditions after statistical and enrichment analysis—preferred selection. log FC (log fold change) indicates the measure of enrichment, a higher value, equals a higher enrichment in the experiments as performed. FDR (false discovery rate) indicates the corrected p-value, a lower value means less variance between replicates as performed.

| Gene | | | | | |
|---|---|---|---|---|---|
| (common | | | | | |
| name, | | | | | |
| SUT or CUT | Name and function | logFC | logFC | FDR | FDR |
| or designation) | (if known) | activation | repression | activation | repression |
| THR 4 | THReonine requiring, | 10.84 | 11.49 | 0.047 | 0.009 |
| | Threonine synthase | | | | |
| MRP10 | Mitochondrial Ribosomal | 10.10 | 6.63 | 0.023 | 0.051 |
| | Protein, Mitochondrial | | | | |
| | ribosomal protein of the | | | | |
| | small subunit | | | | |
| RIP1 | Rieske Iron-sulfur Protein, | 8.47 | 6.65 | 0.037 | 0.058 |
| | Ubiquinol-cytochrome-c | | | | |
| | reductase | | | | |
| YLR342W-A | Gene of unknown function | 9.17 | 7.09 | 0.010 | 0.025 |
| ATG33 | AuTophaGy related, | 10.59 | 11.53 | 0.030 | 0.009 |
| | Mitochondrial mitophagy- | | | | |
| | specific protein | | | | |
| YOR238W | Gene of unknown function | 10.02 | 5.91 | 0.025 | 0.054 |

3. Genes that were Overexpressed—Particularly Preferred Selection

| Gene (common | | | |
|---|---|---|---|
| name, | | | |
| SUT or CUT or | | | |
| systematic | | | |
| designation) | Name and function (if known) | logFC | FDR |
| MIC19 | Component of the MICOS complex | 13.883 | 0.036 |
| TOM22 | Translocase of the Outer Mitochondrial membrane; | 13.781 | 0.008 |
| | responsible for initial import of mitochondrially | | |
| | directed proteins | | |
| NKP1 | Non-essential Kinetochore Protein | 13.389 | 0.012 |
| DML1 | Drosophila melanogaster Misato-Like protein, | 13.307 | 0.014 |
| | Essential protein involved in mtDNA inheritance | | |
| CUT859 | SUT or CUT | 13.152 | 0.033 |
| GAL80 | GALactose metabolism, Transcriptional regulator | 12.170 | 0.008 |
| | involved in the repression of GAL genes | | |
| APM3 | clathrin Adaptor Protein complex Medium chain | 12.088 | 0.020 |
| COQ10 | COenzyme Q, Coenzyme Q (ubiquinone) binding | 12.048 | 0.025 |
| | protein | | |
| BLM10 | BLeoMycin resistance, Proteasome activator | 12.008 | 0.030 |
| MDH1 | Malate DeHydrogenase, Mitochondrial malate | 11.915 | 0.008 |
| | dehydrogenase | | |
| VHS2 | Viable in a Hal3 Sit4 background, Regulator of septin | 11.838 | 0.032 |
| | dynamics | | |
| ASA1 | AStra Associated protein, Subunit of the ASTRA | 11.801 | 0.015 |
| | complex | | |
| TRP4 | TRYPtophan, Anthranilate phosphoribosyl transferase | 11.698 | 0.019 |
| YPS7 | YaPSin, Putative GPI-anchored aspartic protease | 11.620 | 0.030 |
| CUT824 | SUT or CUT | 11.529 | 0.041 |
| YOR318C | Gene of unknown function | 11.515 | 0.013 |
| PRM7 | Pheromone-Regulated Membrane protein | 11.485 | 0.023 |
| ERV46 | ER Vesicle, Protein localized to COPII-coated vesicles | 11.350 | 0.010 |
| FIT2 | Facilitator of Iron Transport, Mannoprotein that is | 11.287 | 0.034 |
| | incorporated into the cell wall | | |
| GPM3 | Glycerate PhosphoMutase | 11.062 | 0.019 |
| CUT892 | SUT or CUT | 10.972 | 0.050 |
| SRN2 | Suppressor of Rna mutations, Number 2 | 10.938 | 0.021 |
| SUT643 | SUT or CUT | 10.910 | 0.039 |
| CUT461 | SUT or CUT | 10.901 | 0.042 |
| THR4 | THReonine requiring, Threonine synthase | 10.840 | 0.047 |
| GMH1 | Gea1-6 Membrane-associated High-copy suppressor; | 10.780 | 0.055 |
| | Golgi membrane protein of unknown function | | |
| SOL1 | Suppressor Of Los1-1, Protein with a possible role in | 10.725 | 0.026 |
| | tRNA export | | |
| NAB6 | Nucleic Acid Binding protein, Putative RNA-binding | 10.674 | 0.013 |
| | protein | | |
| YPR148C | Gene of unknown function | 10.614 | 0.027 |
| ALP1 | Arginine transporter | 10.598 | 0.046 |
| CUT097 | SUT or CUT | 10.597 | 0.046 |
| ATG33 | AuTophaGy related, Mitochondrial mitophagy- | 10.585 | 0.030 |
| | specific protein | | |
| YOR316C-A | Gene of unknown function | 10.547 | 0.025 |
| SOG2 | Key component of the RAM signaling network; | 10.546 | 0.039 |
| | required for proper cell morphogenesis and cell | | |
| | separation after mitosis | | |
| MCM6 | MiniChromosome Maintenance, Protein involved in | 10.531 | 0.019 |
| | DNA replication | | |
| SUT230 | SUT or CUT | 10.507 | 0.010 |
| SUT419 | SUT or CUT | 10.398 | 0.027 |
| TIF11 | Translation Initiation Factor | 10.334 | 0.024 |
| TAF5 | TATA binding protein-Associated Factor, involved in | 10.328 | 0.027 |
| | RNA polymerase II transcription initiation and in | | |
| | chromatin modification | | |
| PHO91 | PHOsphate metabolism, Low-affinity vacuolar | 10.303 | 0.024 |
| | phosphate transporter | | |
| AIM32 | Altered Inheritance rate of Mitochondria, 2Fe-2S | 10.271 | 0.042 |
| | mitochondrial protein involved in redox quality control | | |
| ENO2 | ENOlase, Enolase II, a phosphopyruvate hydratase | 10.260 | 0.050 |
| UBA2 | UBiquitin Activating, Subunit of heterodimeric nuclear | 10.215 | 0.030 |
| | SUMO activating enzyme E1 with Aos1p | | |
| PUS5 | Pseudo Uridine Synthase | 10.197 | 0.030 |
| ERG1 | ERGosterol biosynthesis, Squalene epoxidase | 10.139 | 0.013 |
| SUT311 | SUT or CUT | 10.130 | 0.012 |
| KSS1 | Kinase Suppressor of Sst2 mutations, Mitogen- | 10.116 | 0.039 |
| | activated protein kinase (MAPK) | | |
| MRP10 | Mitochondrial Ribosomal I Mitochondrial | 10.099 | 0.023 |
| | ribosomal protein of the small subunit | | |
| CUT598 | SUT or CUT | 10.099 | 0.046 |
| CUT188 | SUT or CUT | 10.073 | 0.026 |
| YOR238W | Gene of unknown function | 10.023 | 0.025 |
| EMW1 | Essential for Maintenance of the cell Wall, Essential | 15.549 | 0.071 |
| | conserved protein with a role in cell wall integrity | | |
| BNA7 | Biosynthesis of NAD, Formylkynurenine formamidase | 14.863 | 0.071 |
| SNR63 | Small Nucleolar RNA, C/D box small nucleolar RNA | 14.717 | 0.071 |
| | (snoRNA) | | |
| CCT3 | Chaperonin Containing TCP-1, Subunit of the | 14.647 | 0.071 |
| | cytosolic chaperonin Cct ring complex | | |
| PRY2 | Pathogen Related in Yeast, Sterol binding protein | 14.548 | 0.071 |
| | involved in the export of acetylated sterols | | |
| MAL11 | MALtose fermentation, High-affinity maltose | 14.484 | 0.071 |
| | transporter (alpha-glucoside transporter) | | |
| KRS1 | Lysyl (K) tRNA Synthetase | 14.290 | 0.072 |
| RAI1 | Rat1p Interacting Protein, Nuclear decapping | 14.254 | 0.071 |
| | endonuclease | | |
| SUT784 | SUT or CUT | 13.682 | 0.071 |
| YPR148C | Gene of unknown function | 13.572 | 0.071 |
| YEL1 | Yeast EFA6-Like, Guanine nucleotide exchange factor | 13.417 | 0.096 |
| | specific for Arf3p | | |
| CUT832 | SUT or CUT | 13.118 | 0.071 |
| NMA2 | Nicotinamide Mononucleotide Adenylyltransferase | 13.116 | 0.071 |
| VPS27 | Vacuolar Protein Sorting, Endosomal protein that | 12.963 | 0.071 |
| | forms a complex with Hse1p | | |
| SUT428 | SUT or CUT | 12.841 | 0.089 |
| PEX29 | PEroXisome related, ER-resident protein involved in | 12.477 | 0.071 |
| | peroxisomal biogenesis | | |
| YLR446W | Gene of unknown function | 12.369 | 0.071 |
| WBP1 | Wheat germ agglutinin-Binding Protein, Beta subunit | 12.078 | 0.087 |
| | of the oligosaccharyl transferase glycoprotein complex | | |
| AVT2 | Amino acid Vacuolar Transport, Putative transporter | 10.965 | 0.071 |
| CUT854 | SUT or CUT | 10.873 | 0.093 |
| TRM10 | Transfer RNA Methyltransferase, methylates the N-1 | 10.442 | 0.099 |
| | position of guanine at position 9 in tRNAs | | |

Gene to be Preferably Combined with the Particularly Preferred Selection

| | | | |
|---|---|---|---|
| PDI1 | Protein Disulfide Isomerase | 12.524 | 0.072 |

4. Genes or SUTs or CUTs that were Inactivated/Repressed after Statistical and Enrichment Analysis—Particularly Preferred Selection

| Gene (common | | | |
|---|---|---|---|
| name, | | | |
| SUT or CUT or | | | |
| systematic | | | |
| designation) | Name and function (if known) | logFC | FDR |
| TLG2 | T-snare affecting a Late Golgi compartment, Syntaxin- | 13.51 | 0.010 |
| | like t-SNARE | | |
| CUT901 | SUT or CUT | 11.72 | 0.009 |
| ATG33 | AuTophaGy related, Mitochondrial mitophagy-specific | 11.53 | 0.009 |
| | protein | | |
| THR4 | THReonine requiring, Threonine synthase | 11.49 | 0.009 |
| YDR262W | Gene of unknown function | 10.92 | 0.009 |
| CMC1 | Cx9C Mitochondrial protein necessary for full assembly | 10.86 | 0.009 |
| | of Cytochrome c oxidase, Copper-binding protein of the | | |
| | mitochondrial intermembrane space | | |
| MRP17 | Mitochondrial ribosomal protein of the small subunit | 10.20 | 0.019 |
| NDC1 | Nuclear Division Cycle, Subunit of the transmembrane | 14.18 | 0.079 |
| | ring of the nuclear pore complex (NPC) | | |
| PET100 | PETite colonies, Chaperone that facilitates the assembly | 12.69 | 0.086 |
| | of cytochrome c oxidase | | |
| NIP7 | Nuclear ImPort, Nucleolar protein required for 60S | 12.54 | 0.086 |
| | ribosome subunit biogenesis | | |
| VHT1 | Vitamin H Transporter, High-affinity plasma membrane | 12.31 | 0.086 |
| | H+-biotin (vitamin H) symporter | | |
| SUT685 | SUT or CUT | 12.07 | 0.086 |
| BNI5 | Bud Neck Involved, Linker protein responsible for | 11.96 | 0.086 |
| | recruitment of myosin to the bud neck | | |
| SNA3 | Sensitivity to NA+, Protein involved in efficient MVB | 11.93 | 0.086 |
| | sorting of proteins to the vacuole | | |
| EGH1 | Cryptococcus neoformans EGCrP2 Homolog, Steryl- | 11.81 | 0.086 |
| | beta-glucosidase with broad specificity for aglycones | | |
| MRP4 | Mitochondrial ribosomal protein of the small subunit | 11.67 | 0.086 |
| POB3 | PO11 Binding, Subunit of the heterodimeric FACT | 10.87 | 0.086 |
| | complex (Spt16p-Pob3p) | | |
| PIB2 | PtdIns(3)p-Binding, Phosphatidylinositol 3-phosphate | 10.80 | 0.086 |
| | binding protein | | |
| SUT317 | SUT or CUT | 10.74 | 0.086 |
| NTO1 | NuA Three Orf, Subunit of the NuA3 histone | 10.62 | 0.086 |
| | acetyltransferase complex | | |

5. Genes that were Overexpressed—Most Preferred Selection

| Gene (common | | | |
|---|---|---|---|
| name, SUT or | | | |
| CUT or systematic | | | |
| designation) | Name and function (if known) | logFC | FDR |
| MIC19 | Component of the MICOS complex | 13.883 | 0.036 |
| TOM22 | Translocase of the Outer Mitochondrial membrane; | 13.781 | 0.008 |
| | responsible for initial import of mitochondrially | | |
| | directed proteins | | |
| NKP1 | Non-essential Kinetochore Protein | 13.389 | 0.012 |
| DML1 | Drosophila melanogaster Misato-Like protein, | 13.307 | 0.014 |
| | Essential protein involved in mtDNA inheritance | | |
| CUT859 | SUT or CUT | 13.152 | 0.033 |
| GAL80 | GALactose metabolism, Transcriptional regulator | 12.170 | 0.008 |
| | involved in the repression of GAL genes | | |
| APM3 | clathrin Adaptor Protein complex Medium chain | 12.088 | 0.020 |
| COQ10 | COenzyme Q, Coenzyme Q (ubiquinone) binding | 12.048 | 0.025 |
| | protein | | |
| BLM10 | BLeoMycin resistance, Proteasome activator | 12.008 | 0.030 |
| MDH1 | Malate DeHydrogenase, Mitochondrial malate | 11.915 | 0.008 |
| | dehydrogenase | | |
| EMW1 | Essential for Maintenance of the cell Wall, Essential | 15.549 | 0.071 |
| | conserved protein with a role in cell wall integrity | | |
| BNA7 | Biosynthesis of NAD, Formylkynurenine | 14.863 | 0.071 |
| | formamidase | | |
| SNR63 | Small Nucleolar RNA, C/D box small nucleolar RNA | 14.717 | 0.071 |
| | (snoRNA) | | |
| CCT3 | Chaperonin Containing TCP-1, Subunit of the | 14.647 | 0.071 |
| | cytosolic chaperonin Cct ring complex | | |
| PRY2 | Pathogen Related in Yeast, Sterol binding protein | 14.548 | 0.071 |
| | involved in the export of acetylated sterols | | |
| MAL11 | MALtose fermentation, High-affinity maltose | 14.484 | 0.071 |
| | transporter (alpha-glucoside transporter) | | |
| KRS1 | Lysyl (K) tRNA Synthetase | 14.290 | 0.072 |
| RAI1 | Rat1p Interacting Protein, Nuclear decapping | 14.254 | 0.071 |
| | endonuclease | | |
| SUT784 | SUT or CUT | 13.682 | 0.071 |
| YPR148C | Gene of unknown function | 13.572 | 0.071 |
| YEL1 | Yeast EFA6-Like, Guanine nucleotide exchange | 13.417 | 0.096 |
| | factor specific for Arf3p | | |
| CUT832 | SUT or CUT | 13.118 | 0.071 |
| NMA2 | Nicotinamide Mononucleotide Adenylyltransferase | 13.116 | 0.071 |
| VPS27 | Vacuolar Protein Sorting, Endosomal protein that | 12.963 | 0.071 |
| | forms a complex with Hselp | | |
| SUT428 | SUT or CUT | 12.841 | 0.089 |
| PEX29 | PEroXisome related, ER-resident protein involved in | 12.477 | 0.071 |
| | peroxisomal biogenesis | | |
| YLR446W | Gene of unknown function | 12.369 | 0.071 |
| WBP1 | Wheat germ agglutinin-Binding Protein, Beta subunit | 12.078 | 0.087 |
| | of the oligosaccharyl transferase glycoprotein | | |
| | complex | | |

Gene to be Preferably Combined with the Most Preferred Selection

| | | | |
|---|---|---|---|
| PDI1 | Protein Disulfide Isomerase | 12.524 | 0.072 |

6. Genes or SUTs or CUTs that were Inactivated/Repressed after Statistical and Enrichment Analysis—Most Preferred Selection

| Gene (common | | | |
|---|---|---|---|
| name, SUT or | | | |
| CUT or | | | |
| systematic | | | |
| designation) | Name and function (if known) | logFC | FDR |
| TLG2 | T-snare affecting a Late Golgi compartment, | 13.51 | 0.010 |
| | Syntaxin-like t-SNARE | | |
| CUT901 | SUT or CUT | 11.72 | 0.009 |
| ATG33 | AuTophaGy related, Mitochondrial mitophagy-specific | 11.53 | 0.009 |
| | protein | | |
| THR4 | THReonine requiring, Threonine synthase | 11.49 | 0.009 |
| NDC1 | Nuclear Division Cycle, Subunit of the | 14.18 | 0.079 |
| | transmembrane ring of the nuclear pore complex (NPC) | | |
| PET100 | PETite colonies, Chaperone that facilitates the assembly | 12.69 | 0.086 |
| | of cytochrome c oxidase | | |
| NIP7 | Nuclear ImPort, Nucleolar protein required for 60S | 12.54 | 0.086 |
| | ribosome subunit biogenesis | | |
| VHT1 | Vitamin H Transporter, High-affinity plasma membrane | 12.31 | 0.086 |
| | H+-biotin (vitamin H) symporter | | |
| SUT685 | SUT or CUT | 12.07 | 0.086 |

Preferred are further genes or SUTs or CUTs that are selected from the group of genes or SUTs or CUTs having a value of log FC/FDR log FC/FDR of more than 40, preferably of more than 200, more preferred of more than 300, and most preferred of more than 500, based on the values herein.

### REFERENCES AS CITED

1. Martinez Ruiz, J.; Liu, L.; Petranovic, D. (2012) “Pharmaceutical protein production by yeast: towards production of human blood proteins by microbial fermentation”. Current Opinion in Biotechnology, vol. 23(6), pp. 965-971.
2. Falch L A. Industrial enzymes—developments in production and application. Biotechnol Adv. 1991; 9(4):643-58. doi: 10.1016/0734-9750(91)90736-f. PMID: 14542053.
3. Demain A L, Vaishnav P. Production of recombinant proteins by microbes and higher organisms. Biotechnol Adv. 2009 May-June; 27(3):297-306. doi: 10.1016/j.biotechadv.2009.01.008. Epub 2009 Jan. 31. PMID: 19500547.
4. Zahrl R J, Gasser B, Mattanovich D, Ferrer P. Detection and Elimination of Cellular Bottlenecks in Protein-Producing Yeasts. Methods Mol Biol. 2019; 1923:75-95. doi: 10.1007/978-1-4939-9024-5_2. PMID: 30737735
5. Parapouli M, Vasileiadis A, Afendra A S, Hatziloukas E. Saccharomyces cerevisiae and its industrial applications. AIMS Microbiol. 2020 Feb. 11; 6(1):1-31. doi: 10.3934/microbiol.2020001. PMID: 32226912; PMCID: PMC7099199.
6. Dominguez A A, Lim W A, Qi L S. Beyond editing: repurposing CRISPR-Cas9 for precision genome regulation and interrogation. Nat Rev Mol Cell Biol. 2016 January; 17(1):5-15. doi: 10.1038/nrm.2015.2. Epub 2015 Dec. 16. PMID: 26670017; PMCID: PMC4922510.

1. A cell of Saccharomyces cerevisiae, producing at least one secreted protein of interest, wherein at least one of:
the cell comprises at least one fungal gene selected from the group consisting of ENO2, NMA2, PRY2, SUT074, TFG2, AVT2, TRM10, BNA7, and TOM22, wherein said at least one fungal gene shows at least one of increased expression and overexpression; and
the cell comprises at least one further fungal gene selected from the group consisting of TLG2, MNT2, TPO2, ATG33, THR4, INP51, CUT901, YDR262W, MRP10, NDC1, and CMC1, wherein the at least one further fungal gene shows at least one of reduced expression and inactivation.

2. The cell according to claim 1, wherein the cell further comprises at least one of:
a still further fungal gene selected from the groups consisting of ENO2, NMA2, PRY2, SUT074, and TFG2, or AVT2, TRM10, PRY2, SUT074, BNA7, and TOM22, wherein the still further fungal gene shows at least one of increased expression and overexpression; and
at least one additional fungal gene selected from the groups consisting of TLG2, CUT901, ATG33, THR4, YDR262W, and CMC1, or MRP10, TLG2, CUT901, ATG33, THR4, YDR262W, CMC1, MNT2, TPO2, and NDC1, wherein the at least one additional fungal gene shows at least one of reduced expression inactivation; and
one or more of at least one of fungal genes HDA2 PDI1 that show at least one of an increased expression and overexpression and fungal gene INP51 showing at least one of reduced expression and inactivation.

3. The cell according to claim 1, wherein the genes have a value of log FC/FDR log FC/FDR of more than 40.

4. The cell according to claim 1, wherein the cell is from Saccharomyces cerevisiae strain ER.sec2.

5. The cell according to claim 1, wherein said at least one secreted protein of interest also shows at least one of an increased expression and overexpression.

6. The cell according to claim 1, wherein one or more of the fungal genes are one of native genes and recombinant genes, wherein each of the recombinant genes is one or more of integrated into the genome as an expression cassette and extrachromosomally expressed using a replicative expression vector.

7. The cell according to claim 6, wherein the cell further comprises at least one additional recombinant secretion promoting gene comprising a gene for a chaperone, for a foldase and for a glycosylation-promoting protein.

8. The cell according to claim 7, wherein one or more of the increased expression, overexpression, reduced expression, and inactivation of one or more of the at least one fungal gene and the at least one additional recombinant secretion promoting gene are one of constitutive and inducible.

9. The cell according to claim 1, wherein the cell produces the at least one secreted protein to at least 30% more than a control yeast or filamentous fungal cell.

10. A method for producing a secreted protein in a cell, comprising the steps of i) providing a cell of Saccharomyces cerevisiae according to claim 1, producing at least one secreted protein of interest; ii) culturing the cell in suitable culture medium.

11. The method according to claim 10, wherein at least 30% more of the at least one secreted protein is produced, when compared to a production of a control cell.

12. A method for producing a yeast cell producing at least one secreted protein of interest, comprising introducing into the cell producing at least one secreted protein of interest at least one fungal gene selected from the group consisting of ENO2, NMA2, PRY2, SUT074, TFG2, AVT2, TRM10, BNA7, and TOM22, wherein the at least one fungal gene shows at least one of increased expression and overexpression, and/or wherein the cell comprises at least one further fungal gene selected from the group consisting of TLG2, MNT2, TPO2, ATG33, THR4, INP51, CUT901, YDR262W, MRP10, NDC1, and CMC1, wherein the at least one further fungal gene shows at least one of reduced expression inactivation.

13. The method according to claim 12, wherein the at least one fungal gene is at least one of integrated into the genome as an expression cassette and extrachromosomally expressed using a replicative expression vector.

14. (canceled)

15. The method according to claim 10, further comprising suitably inducing the increased expression, overexpression, reduced expression, and inactivation of the at least one fungal gene in the cell.

16. The method according to claim 12, further comprising introducing into the cell a fungal gene selected from the group consisting of RIP1, YLR342W-A, and YOR238W that shows one or more of an increased expression, overexpression, reduced expression, and inactivation depending on experimental conditions.

17. The method according to claim 16, further comprising introducing into cell at least one of fungal gene HDA2 and PDI1 that show at least one of an increased expression and overexpression.

18. The cell according to claim 1, wherein the cell further comprises at least one of fungal genes HDA2 and PDI1 that show at least one of an increased expression and overexpression.
